(19) United States
(12) Patent Application Publication
(10) Pub. No.: US 2006/0122865 A1
(43) Pub. Date: Jun. 8, 2006

(54) Procedural medicine workflow management

(76) Inventors: Erik Preiss, Underhill, VT (US); Kimberly Stavrinakis, Richmond, VT (US); Ronald Keen, Shelburne, VT (US); Debra Stenner, Ferrisburgh, VT (US)

Correspondence Address: FENWICK & WEST LLP, SILICON VALLEY CENTER, 801 CALIFORNIA STREET, MOUNTAIN VIEW, CA, 94041, US

(21) Appl. No.: 11/283,417

(22) Filed: Nov. 17, 2005

Related U.S. Application Data
(60) Provisional application No. 60/630,879, filed on Nov. 24, 2004.

(51) Int. Cl. G06Q 10/00; G05B 19/418

(52) U.S. Cl. 705/2; 705/8

(57) ABSTRACT

A procedural medicine workflow system collects data relating to medical procedures, organizes workflows, and allows statistics to be gathered on such procedures. The system includes a data management subsystem, a span of procedure subsystem, a work organization subsystem, and a business management subsystem. The system allows information from disparate sources to be used via a common vocabulary.

### CROSS-REFERENCE TO RELATED APPLICATIONS

This application claims the benefit of U.S. Provisional Application No. 60/630,879, entitled “Procedural Medicine Workflow”, filed Nov. 24, 2004, which is incorporated by reference herein in its entirety.

This application is related to non-provisional Application Ser. No. 10/301,404, filed Nov. 20, 2002, entitled “Autogeneration of Patient Information”, which is incorporated by reference herein.

### BACKGROUND

1. Field of the Invention

This invention relates generally to the field of healthcare information management, and more particularly to the management and integration of information necessary for the optimization of efficiencies in the service lines that utilize imaging-based procedures.

2. Background Art

As the use of procedure-based medicine grows, the need for improving specialty department efficiencies and outcomes has become more apparent. Many healthcare institutions are still using paper-based systems or non-integrated electronic systems to manage their work. This results in a continued need for clinical staff to locate information or images that are needed in the process of providing patient care. Providing a single view of the patient across specialties and a “deep” view within each specialty is needed to significantly improve patient care that depends on medical procedures.

Procedure-based medicine, e.g., cardiology, oncology, gastroenterology, general surgery, differs in a number of important aspects from primary care medicine, e.g., family practice, pediatrics, geriatrics, general internal medicine. The latter, which are typically more general practices, encompass an enormously wide range of activities, from preventive physical examinations to disease management to treatment of common injuries. The breadth of such practices has led to systems for increasing practice effectiveness to be based on a patient-centric view of workflows, as can be found in various electronic medical records (EMR) systems and hospital information systems (HIS's). This patient-centric view is evident, for example, in U.S. Pat. No. 5,974,389 to Clark et al. for Medical Record Management System and Process with Improved Workflow Features, which describes “A patient medical record system” having “a patient record database . . . ” Patient-centric workflows made sense when most medical services were provided by attending physicians and other caregivers at the patient's bedside.

In recent years, more and more health care has been provided through procedure-based medicine. This type of health care is characterized by more strictly defined activities than one might find in a general medical practice, and the bulk of activity in such practices may not involve real-time interaction with the patient. For example, the bulk of activity in a typical radiology practice might not come during the actual patient interaction, but in preparation for the patient visit (e.g., based on reports from other healthcare providers) or in subsequent analysis of the images obtained during the patient visit. Such a radiology practice might primarily include conventional x-ray imaging, computed tomography (CT) three-dimensional scans, ultrasound procedures, mammograms and magnetic resonance imaging scans as the five modalities that make up the bulk of the diagnostic imaging side of the practice. Likewise, there may be a relatively small number of procedures that make up the interventional side of the practice. For a typical diagnostic imaging practice, the tasks for each type of imaging are fairly well defined, from patient intake through diagnosis and reporting. Given the expected constraints on these procedures, and the limited part of the procedures that involve real-time interaction with the patient, the use of patient-centric workflow systems may be less efficient than systems centered on the procedures themselves.

Known healthcare information systems and related solutions, such as those provided by IDX Systems Corporation of South Burlington, Vt., address management of information ranging from medical test results to insurance information. Focusing on one specific example, modem medical image management systems typically consist of a Picture Archiving and Communication System (PACS) interfaced to a Radiology Information System (RIS). PACS systems are generally implemented such that the patient demographic, order data, and result data are managed by a single RIS. The interfaces are configured such that the PACS receives patient demographics, exam order data, and exam result data via unsolicited interface transactions transmitted by the RIS. The implementation is designed to handle the following expected standard workflow:
A user enters patient demographics into the RIS. A required portion of the patient demographics is a unique Patient ID used to identify the patient.
The RIS sends an interface transaction containing the patient demographics including the Patient ID to the PACS.
The PACS receives the transaction from the RIS and stores the patient demographics in the PACS database.
A user orders or schedules an exam in the RIS for this patient. The RIS creates an Accession Number that is used to uniquely identify the exam.
The RIS sends an interface transaction containing both the patient demographics including the Patient ID and the exam order information including the Accession Number to the PACS.
The PACS receives the transaction from the RIS and stores the exam order information in the PACS database. The PACS uses the Patient ID provided in the interface transaction to associate this exam to the correct patient.
A user performs the exam on the patient using an imaging modality. If the imaging modality supports a method for patient and exam demographic download, the modality will receive the demographic data directly from the RIS. Otherwise, the user enters the patient demographics and exam data including at least the Patient ID and the Accession Number into the user interface provided by the imaging modality.
The imaging modality acquires medical images and sends the study (i.e., images and related information) with the patient demographics and exam data to the PACS.
The PACS receives the study and extracts the patient demographic and exam information from the image headers and uses the Patient ID and Accession Number to match the study with the information previously filed in the PACS database. Users of the PACS can now view images for this exam. Since the study has been reconciled to the exam, the users can access the patient's record using this accurate information.
A user manually enters information into the RIS to denote that the exam has been performed. Users of the RIS can now see that the exam for this patient has been performed and is complete. The status of the exam in the RIS reflects that it is ready to be interpreted.

Oftentimes, real-world healthcare institutions having PACS and other medical image management systems receive data from both internal and external sources. Internal sources of data include the RIS and other information systems owned and/or managed by the healthcare institution operating the medical image management system. External sources of data generally include imaging equipment and other information systems that are owned and/or managed by different healthcare institutions. In general, different healthcare institutions use different numbering schemes for identifying patients and exams. Hence, data received from external sources generally do not directly correspond with data provided by internal sources.

For example, a patient may have several radiology exams performed at a local medical center, which are interpreted by physicians at that center. The medical image management system at the medical center has all of the demographic information, examination information, and medical images for this patient. If the patient subsequently has a radiology exam performed at a clinic across town that is owned and/or managed by an entity other than the medical center, but needs to have the images interpreted by a radiologist at the medical center, then the images are electronically transferred to the medical center. When the images are transferred from the clinic's image management system to the medical center's image management system, it is very likely that the identifiers for the patient and exam information in the images do not correspond to the data in the medical center's image management system. Thus, it is difficult to determine the patient to which the images are related and errors may be introduced into the center's image management system.

One existing approach to this issue is for the center's system to indiscriminately accept images and present all data as valid regardless of its source. This eliminates the visibility of the problem. However, this scheme presents a problem because the user of the system either doesn't know any better and assumes that all data is valid, or if the user understands that data may be inaccurate, the user assumes all data is suspect and takes extra time to verify it. In the first case where the user assumes that all data is valid, there is a significant risk of medical errors. In the worst case scenario, if incorrect information displayed in the PACS for a set of images, there could be a delay in diagnosis or even a lack of diagnosis and treatment for the correct patient. In the second case where the user takes extra time to reconcile and verify that the data are accurate, there will be a delay in patient care.

A second approach that addresses this issue is to treat any set of images that arrives at the PACS that cannot be reconciled with patients and/or exams in the PACS database as a “broken” study. The PACS generally supplies functionality for a system administrator to fix these “broken” studies by either matching a study to a patient and exam that already exists in the PACS database or by manually creating the patient and exam record, then reconciling the study to the newly created exam. There is much less risk of resulting with inaccurate data than the first approach since a human user interacts with the system to verify a match. However, since the process for resolving “broken” studies is detached from the normal process, productivity is reduced and results in delay of diagnosis and delay of necessary treatment of those patients who have “broken” studies. Again, in the worst case, it is possible that this delay could cause the death of a patient.

A third approach that addresses this issue is to implement the PACS such that it is restricted to only receive images from known sources that are managed by the RIS that it is connected to. If the customer desires to acquire images from a source that is managed by a RIS that is not connected to the PACS, the customer must either convert data from the new source to their RIS or manually enter the data in both information systems. While this solution provides the most accurate patient information, it is impractical and in the case of a conversion, is very costly.

Recognizing that healthcare information systems often do not stand alone but are preferably interconnected, some attempts have been made to facilitate transfer of information from one system to another. For example, in 1987 an effort known as Health Level Seven (HL7) was initiated that resulted in a series of standards for the exchange of electronic information in healthcare environments. The HL7 protocol enables diverse users such as hospital information systems, clinical laboratory systems, and pharmacies to exchange information in a manner most helpful to each.

As noted above, however, not all information arriving at a facility from an external source, or possibly even from an internal source, will be in compliance with an HL7 or another standard. In these situations, where patient data from one source does not match up perfectly with patient data from another source, it can be difficult and time consuming to appropriately reconcile the information for subsequent use. Previous solutions to this problem involved exception handling for such “broken studies” or similar techniques that route such studies to special interfaces or facilities for reconciliation, whether completely manual or with some electronic facilitation. For example, the PACS Broker product from AGFA includes a facility for linking PACS information to Radiology Information System (RIS) information. Mismatches in patient information are essentially filed in a special “bucket” for broken studies.

Continuing with just this one example, inefficiencies arise because none of the currently known systems adequately processes such broken studies so as to take full advantage of redundancies in patient information from diverse sources. In this instance, therefore, there is a need for a way to more effectively handle interchange of electronic patient information from diverse non-standardized sources, not all of which conform to an existing interchange standard.

More generally, there is a need to increase caregiver productivity by addressing workflows from the perspective that is most pertinent to the medical procedure being performed. In some instances, it may remain preferable to maintain a patient-centric workflow analysis. As procedural medicine becomes more the norm, however, it will be more common that workflow is addressed based not on the viewpoint of information about a particular patient, but on the viewpoint of information concerning the procedure to be performed.

Some known solutions address a small portion of the overall workflow related to a procedure; for instance hemodynamic systems from Witt Biomedical Corporation, of Melbourne, Fla., and General Electric (GE) Corporation, of Fairfield, Conn.

Witt Biomedical Corporation and GE Corporation address the in-lab portions of a cardiology procedure but do not address the pre and post-lab procedural steps. However, no solution is known that attempts to increase overall procedural medicine workflow productivity, or to comprehensively address a collection of workflow elements that relate to particular procedures. For example, known systems that are patient-centric typically focus on only the final result of a procedure and not the procedure itself, and thus do not capture the full depth of data available from the procedure. They do not address data mining available from monitoring procedures across patient populations, or the trending statistics that can be generated as a result. They do not typically address procedure-specific physician and staff efficiencies, as once again such issues largely extend far beyond single-patient data, for instance the issues of facilitating physician office access to hospital-generated information concerning procedures.

The goal of improving clinical outcomes while simultaneously reducing the cost of care delivery has the majority of provider institutions focused on implementing several core clinical systems: CPOE, pharmacy, medical guidelines/decision support, multi-disciplinary documentation, and clinical repositories for the centralized storage of all actions taken and the associated outcome. Controlled medical vocabularies, and even to some degree document imaging, are supporting technologies that will enable a more comprehensive and seamless view across these core systems. Current thinking would cause institutions buying today to purchase these core systems, including ADT and master person indexing capabilities, from a single vendor.

It is advantageous for these core systems to be “enterprise” in nature, e.g. able to provide comprehensive longitudinal clinical histories (womb to grave) for a given patient population, in order to deliver on the goal of improving outcomes while reducing costs. Toward that end, they should be very broad-based, accommodating every medical specialty, and accessible from virtually any point in the institution. These core systems should be able to cover both inpatient and outpatient encounters, and be able to cross-organizational boundaries when providing services to outlying non-owned entities (business-to-business capabilities). Developing and deploying these “enterprise” systems is a major undertaking for both the vendor and the consumer.

At the same time that health care institutions are striving to implement these overarching “enterprise” solutions, the growth of procedural based medicine is exploding. This growth in procedural medicine is fueled by advances in medical technology, an increase in sub-specialization, and by an increase in demand due to global population growth and the “graying of America”. The evolution of medical technology has resulted in an increasing number of new diagnostic and/or interventional procedures. Nearly all procedure-based medicine relies heavily on medical imaging, underscoring the need for efficiencies in this area.

### DISCLOSURE OF THE INVENTION

In accordance with the present invention, a procedural medicine workflow system collects, filters, and disseminates medical data necessary for each stage of a medical procedure to the appropriate caregiver at the appropriate time.

The procedural medicine workflow system includes a data management subsystem, a span of procedure subsystem, a work organization subsystem, and a business management subsystem. The data management subsystem captures data needed to perform, manage, and measure patient care within a department.

The span of procedure subsystem includes components that cover management of the entire life cycle of a particular medical procedure. The span of procedure subsystem captures insurance information, medical orders, and history and ultimately returns results to the physician's office.

The work organization subsystem addresses different needs of various specialists and staff throughout a procedure by tailoring the data collected from disparate sources to meet the specific needs of the specialists and staff. The work organization subsystem takes subsets of data from the data management subsystem and, at the appropriate point in the procedure, presents it in a manner most suitable for the professional who needs such data at that time.

The business management subsystem includes components to monitor and measure business aspects and patient care aspects of the specialty departments corresponding to a particular procedure, so as to facilitate optimization of both throughput and patient outcomes.

In accordance with the present invention, the procedural medicine workflow system optimizes throughput of the procedural department and manages efficiencies of the procedural department through continual monitoring and reallocation of resources.

Further, in accordance with the present invention, the procedural medicine workflow manages its components to facilitate re-use of such components for various workflows across multiple specialty-care settings.

The features and advantages described in this summary and the following detailed description are not all-inclusive. Many additional features and advantages will be apparent to one of ordinary skill in the art in view of the drawings, specification, and claims hereof. Moreover, it should be noted that the language used in this disclosure has been principally selected for readability and instructional purposes, and may not have been selected to delineate or circumscribe the inventive subject matter, resort to the claims being necessary to determine such inventive subject matter.

### BRIEF DESCRIPTION OF THE DRAWINGS

FIG. 1 is a high-level block diagram illustrating a procedural medicine workflow system according to one embodiment of the present invention;

FIG. 2 illustrates an application structure according to one embodiment;

FIG. 3 illustrates a procedure-centric workflow as managed by the system of FIG. 1;

FIG. 4 illustrates integration of reporting tools by the system of FIG. 1;

FIG. 5 illustrates a processing sequence performed by various components of the system of FIG. 1;

FIG. 6 is a high-level block diagram illustrating a medical database system with capabilities for autogeneration of patient information according to one embodiment of the present invention;

FIG. 7 is a high-level block diagram of a computer system for use as the medical database system of FIG. 6 according to one embodiment;

FIG. 8 is a flowchart illustrating the operation of the patient matching module of the comparison subsystem in the medical database system;

FIG. 9 is a flowchart illustrating steps performed by the provider matching module when performing provider matching processing according to an embodiment of the comparison subsystem;

FIG. 10 is a flowchart illustrating steps performed by the study matching module when performing study matching processing according to an embodiment of the comparison subsystem;

FIG. 11 is a flowchart illustrating the operation of the autogeneration subsystem according to an embodiment of the medical database system;

FIG. 12 is a flowchart illustrating the operation of the mapping tool subsystem according to one embodiment of the medical database system; and

FIG. 13 is a block diagram illustrating a more detailed view of the dictionary subsystem.

FIG. 14 is a user interface that allows a user to configure a worklist according to an embodiment of the present invention.

FIG. 15 is a user interface that allows a user to select columns to be displayed in the worklist.

FIG. 16 shows a worklist created by a user.

The figures depict an embodiment of the present invention for purposes of illustration only. One skilled in the art will readily recognize from the following description that alternative embodiments of the structures and methods illustrated herein may be employed without departing from the principles of the invention described herein.

### DETAILED DESCRIPTION OF THE PREFERRED EMBODIMENTS

While the present invention will be described in connection with preferred embodiments thereof, it will be understood that it is not intended to limit the invention to those embodiments. On the contrary, it is intended to cover all alternatives, modifications, and equivalents as may be included within the spirit and scope of the invention as defined by the appended claims.

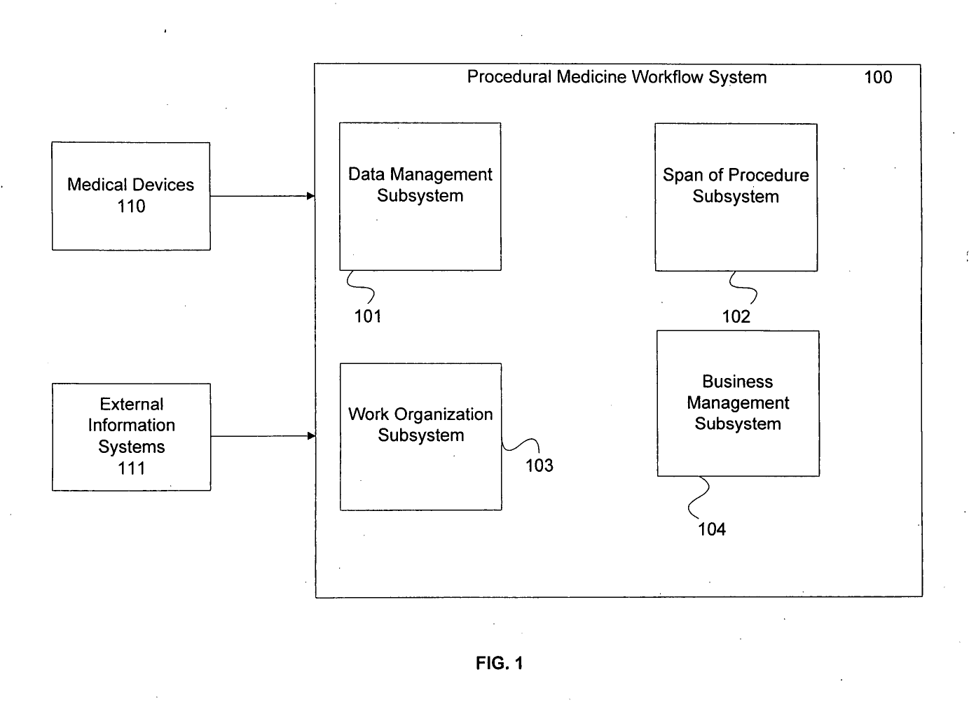

FIG. 1 is a high-level block diagram illustrating a procedural medicine workflow system 100 according to one embodiment of the present invention. System 100 is implemented as a web-based software system providing a single mechanism for monitoring and improving clinical outcomes and business efficiencies of hospital service lines. The discussion that follows focuses on radiology, but those skilled in the art will appreciate that similar approaches are equally applicable to other procedural medicine practice areas.

In system 100, workflow is addressed from a “procedure-centric” point of view, so that the various resources that are needed to perform a procedure have the information they need readily available at the time it is needed.

According to one embodiment of the present invention, the primary functional components of system 100 are a data management subsystem 101, a span of procedure subsystem 102, a work organization subsystem 103, and a business management subsystem 104.

Data management subsystem 101 captures the detailed data needed to perform, manage and measure patient care within a department. In one embodiment, subsystem 101 includes digital image acquisition and display, medical device integration, electronic medical record integration, scanned documents and structured clinical data gathering. Data from each of these components is gathered conventionally and managed as described below by subsystem 101.

Span of procedure subsystem 102 includes components that cover management of the entire life cycle of a particular medical procedure. In one embodiment, this coverage includes data capture from a physician's office concerning information such as insurance, orders, and history, continues through intra-department efficiencies such as scheduling and tracking, and ultimately returns results to the physician's office.

Work organization subsystem 103 addresses different needs of various specialists and staff throughout a procedure by tailoring the data collected from disparate sources to meet the specific needs of the specialists and staff. Thus, this subsystem takes subsets of data from data management subsystem 101 and, at the appropriate point in the procedure, presents it in a manner most suitable for the professional who needs such data at that time.

Business management subsystem 104 includes components to monitor and measure business aspects and patient care aspects of the specialty departments corresponding to a particular procedure, so as to facilitate optimization of both throughput and patient outcomes.

According to one embodiment of the present invention, system 100 integrates with medical devices 110 and external information systems 111. In a preferred embodiment, a set of standard web services allowing communication among disparate applications, such as Service Oriented Architecture (SOA) technology provided by IDX Systems Corporation, is used to accomplish this integration. In alternate embodiments, other integration mechanisms used with system 100 include messaging standards such as DICOM, HL7, HTTP and X12, depending on the particular procedures involved and the prevalent health industry standards for such procedures.

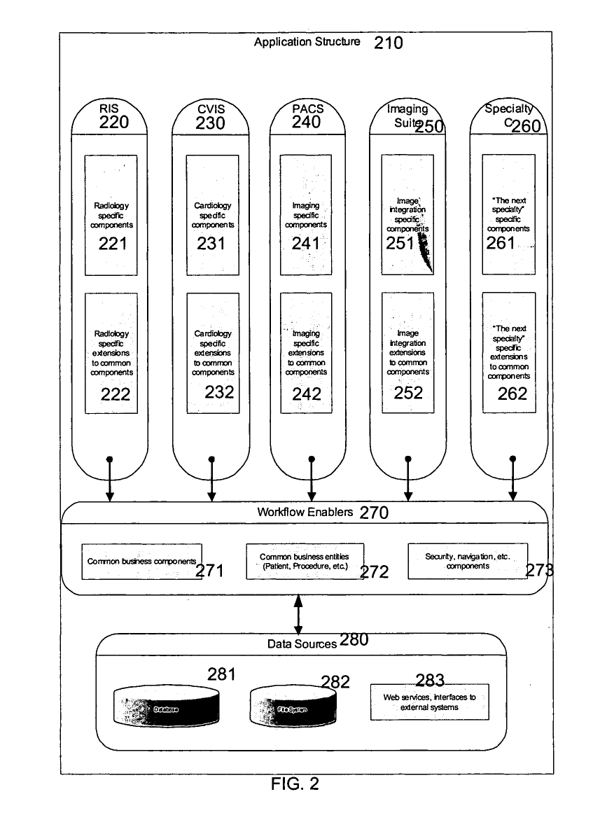

Referring now to FIG. 2, according to one embodiment, system 100 is implemented using a web-based application structure 210 in an n-tiered architecture. Specifically, application structure 210 addresses five imaging-related specialties: RIS (radiology) 220, CVIS (cardiology) 230, PACS (picture archiving) 240, Imaging Suite (a medical imaging management system available from IDX Systems Corporation of South Burlington, Vt.) 250, and “Specialty C” (a generic reference to additional aspects for future specialties as may be needed by a hospital or other practice) 260. Each of these has their own specific components (221, 231, 241, 251, and 261) and extensions (222, 232, 242, 252, and 262). For example, component 221 includes functionality for tracking and reporting on mammography procedures which are not utilized by the other specialties. Component 232 includes functionality for capturing data elements that are specifically required for cardiology cath lab certification. Component 222 uses similar data collection methodology for angiography exams. One skilled in the art would understand that the data elements themselves in component 222 are specific to the specialty.

Shared by all of these specialties are common workflow issues, handled by workflow enablers 270. In a preferred embodiment, these include common business components 271, such as procedure scheduling, common business entities 272 that are the data schemas used by business components, and components 273 to handle global tasks such as security and navigation within system 100. As one specific example, patient-centric systems typically support role-based security (e.g., granting permission for various tasks to be performed using the system) at the organization level. However, when multiple specialties use the same information system, such security is not sufficiently granular. There is no need, for instance, for a cardiac catheterization lab technician to be able to edit data associated with a mammography examination. In a preferred embodiment, components 273 provide such role-based security.

Other enablers 270 not shown in FIG. 2 include common workflow components, i.e., components that were developed for one procedure but that may be re-used in workflows for other procedures; base business/workflow components, which are fundamental components provided as a “toolkit” for workflow management; and common integration components, used to establish correspondences among various roles, organizations, and people as described herein.

Workflow enablers 270 are in communication with various data sources 280, which can be categorized as existing databases 281, file system data 282, and external sources 283 such as web services connecting to other systems and other types of interfaces to external systems.

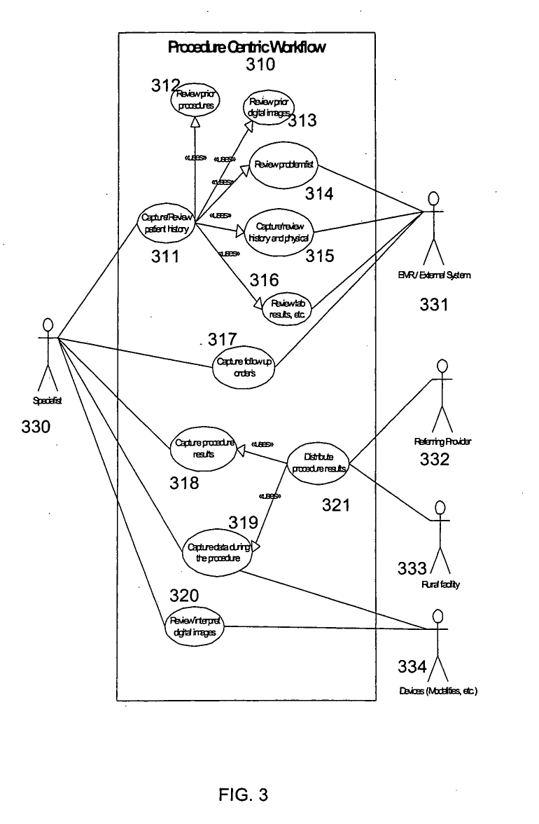

Turning now to FIG. 3, there is illustrated one example of procedure-centric workflow management in accordance with the present invention. This example manages a workflow involving a specialist 330, an electronic medial record (EMR) or other external patient information system 331, a referring provider 332, a rural health care facility 333, and appropriate devices 334 (e.g., modalities) corresponding to the particular procedure of interest. In this example, the specialist's workflow includes capturing/reviewing patient history 311, which itself entails reviewing prior procedures 312, reviewing prior digital images 313, reviewing problem lists 314 in communication with EMR 331, capturing/reviewing patient physical and history information in communication with EMR 331, and reviewing lab results in communication with EMR 331. The workflow further includes capturing follow-up orders 317 in communication with EMR 331. In addition, the workflow also involves capturing procedure results 319 and corresponding data obtained during the procedure 320, and distributing 321 such data in communication with a referring provider 332 and rural facility 333. Finally, in communication with external devices 334, specialist 330 receives the captured data from the procedure 319 and reviews/interprets the digital images 320. Workflow management as described here includes recognition of various roles of people involved in workflows, whether they are different types of caregivers or different types of patients. Thus, as the overall range of workflows being managed increases, the user is not presented with information that is irrelevant to the context of the user's current need. For example, an echo cardiologist reading stress echoes may not need to be presented with interventional cardiology features, even if such features are available.

In preferred embodiments, system 100 is configurable for operation in several different modes. System 100 is configurable for use as a standalone department system. It is also configurable for use as a departmental system integrated into third party portals and frameworks permitting, for example, policies of a hospital to be taken into account along with policies of physician groups performing procedures in the hospital, or policies of different specialties within a hospital to be addressed. In one implementation of system 100, a diagnostic report and professional bill reflects the logo and address of the provider group an attending physician belongs to, and not necessarily just the hospital where a procedure was performed. Such integration can be further extended to third party portals and frameworks covering multiple health service lines. Another configuration of system 100 is as a standalone enterprise application covering multiple health service lines.

Note that system 100 is configured to differentiate “procedures” from traditional “examinations”. Known systems focus on the latter, which primarily involve a single test that is performed, with a report of the results following. Procedures, on the other hand, are more generalized and involve a series of steps that occur in order to treat or diagnose an ailment, which in turn can generate multiple results. Thus, an examination may be considered the simplest of a procedure, having only a single step. Support for more generalized procedures as described herein more completely addresses typical workflows concerning, e.g., Radiology and Cardiology IHE profiles, SPS/MPSS and specialty scheduling (staff and resource scheduling).

Even simple procedures differ from the concept of a patient visit. For example, if a patient is undergoing an interventional cardiac catheterization and there is a need to perform an intravascular ultrasound, then depending on which department owns the equipment and the credentials of the attending physician there may be a need to result and bill the ultrasound separately from the cardiac catheterization. System 100 is configured to consider such an ultrasound as an episode of care and keep it associated with the catheterization, rather than treating it separately in the conventional manner as a new patient visit. By maintaining such associations, system 100 avoids redundant storage of information that appears unrelated but really is related. System 100, by maintaining the correspondences within procedures, allows the user to cross-reference and get details on all the steps that went into a particular procedure for a particular patient. Furthermore, by keeping accounts of such associations, system 100 provides drag-and-drop scheduling and task assignment for any required portion of a procedure, without regard to whether the corresponding resources are from one practice group or different practice groups.

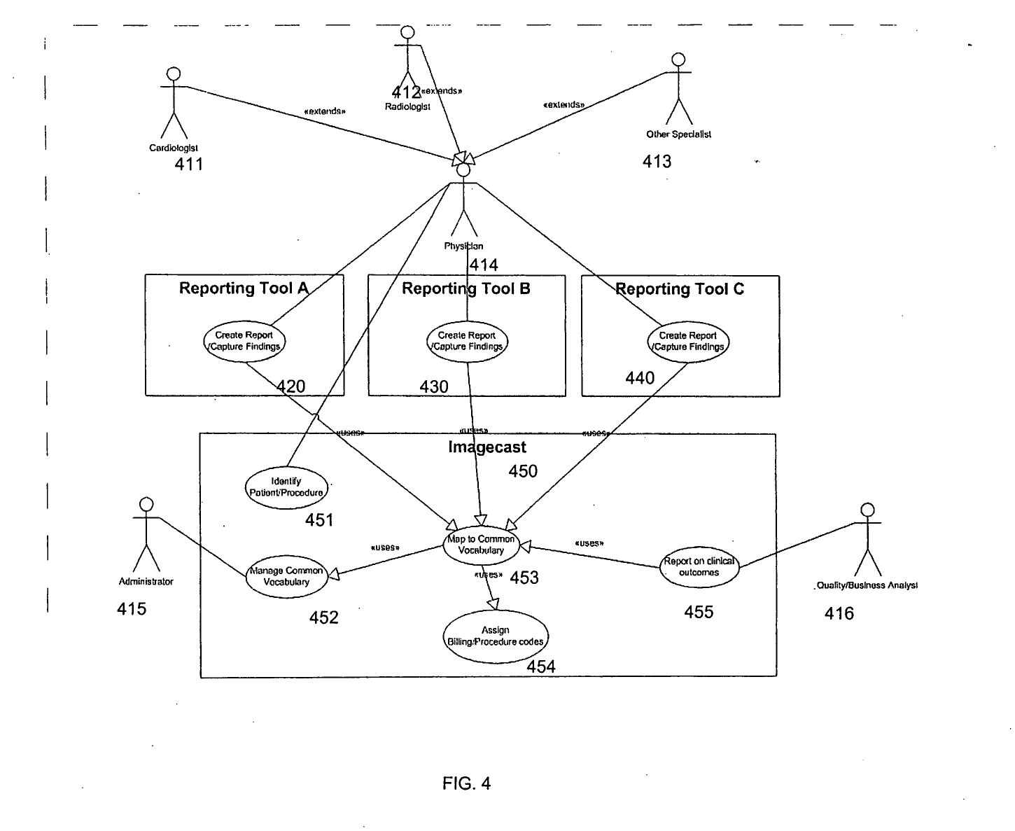

Referring now to FIG. 4, a functional block diagram is provided showing how system 100 operates to integrate disparate reporting tools 420, 430, 440, with a medical image management system (for example the Imagecast system provided by IDX Systems Corporation of South Burlington, Vt.) 450. Specialist health care providers, such as cardiologist 411, radiologist 412 or other specialist 413 all communicate with a patient's primary physician 414 to provide patient care. Various specialties typically have their own reporting tool, e.g., 420, 430, and 440, for capturing procedural findings and creating corresponding reports. As described herein, system 100 allows a user (physician 414 in this instance) to identify 451 a patient or procedure and, via an image management system 450, combine and coordinate such various reporting tools 420, 430, and 440. Specifically, system 100 via image management system 450 maps 453 the reports created by tools 420, 430, 440 into a common vocabulary, allows the common vocabulary to be managed (updated and revised as needed) 452 by an administrator 415, facilitates assignment of billing and procedure codes 454, and permits a quality or business analyst 416 to work with and generate reports on clinical outcomes 455 (e.g., trend reports, efficiency reports) for overall improvement of patient procedures.

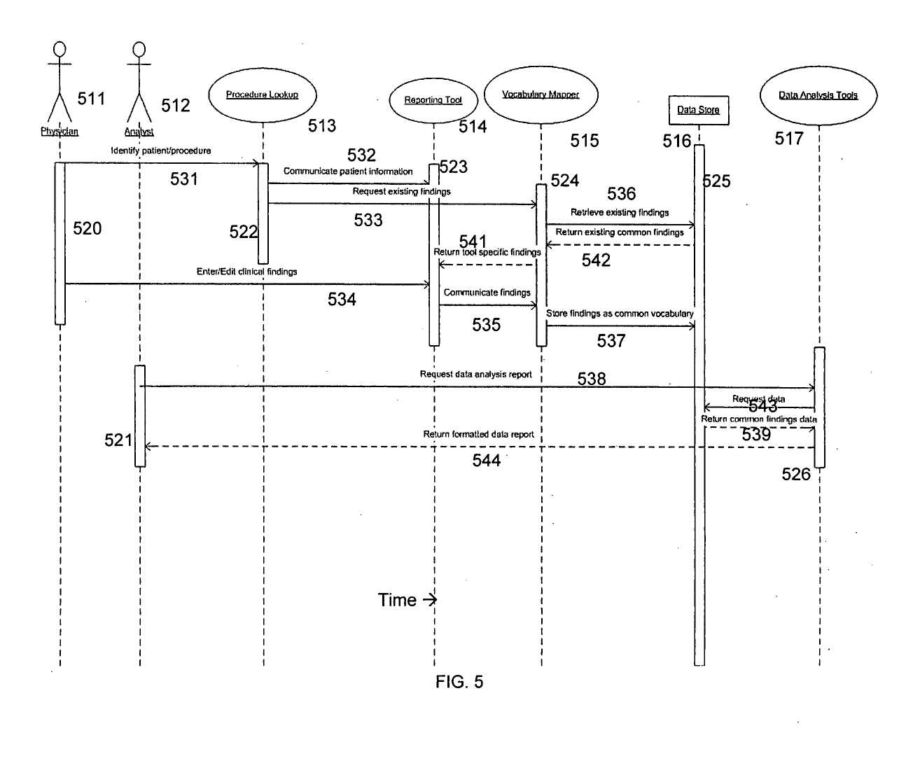

Referring now to FIG. 5, there is shown a processing sequence for workflow processing in accordance with the present invention. In this instance, two caregivers are involved—physician 511 and analyst 512. In FIG. 5, the above entities are listed across the top. Beneath each entity is a vertical line representing the passage of time. The horizontal arrows between the vertical lines represent transactions between the associated entities. It should be noted that not every transaction is shown in FIG. 5. In other embodiments of the present invention, the order of the transactions can vary.

At stage 520, system 100 permits physician 511 to identify a patent/procedure of interest 531 and begin entering or editing clinical findings 534. At stage 522 when the patient or procedure is identified, system 100 then undertakes a procedure lookup 513, based on which it communicates patient information 532 and requests existing findings 533. At stage 523, when the patient information is accessed and the clinical findings are entered/edited, reporting tool 514 begins to communicate findings. At stage 524, after the request for findings 523 is processed and the findings are communicated 535, vocabulary mapper 515 returns tool-specific findings to reporting tool 514. Once vocabulary mapper 515 has the information it needs, it retrieves existing findings 536 and stores findings as common vocabulary 537 in data store 516. Back at stage 521, analyst 512 requests a data analysis report 538; at stage 526, data analysis tools 517 are able to request 543 such information from data store 516, the common findings data are returned 539, and the formatted data report 544 is returned to analyst 512.

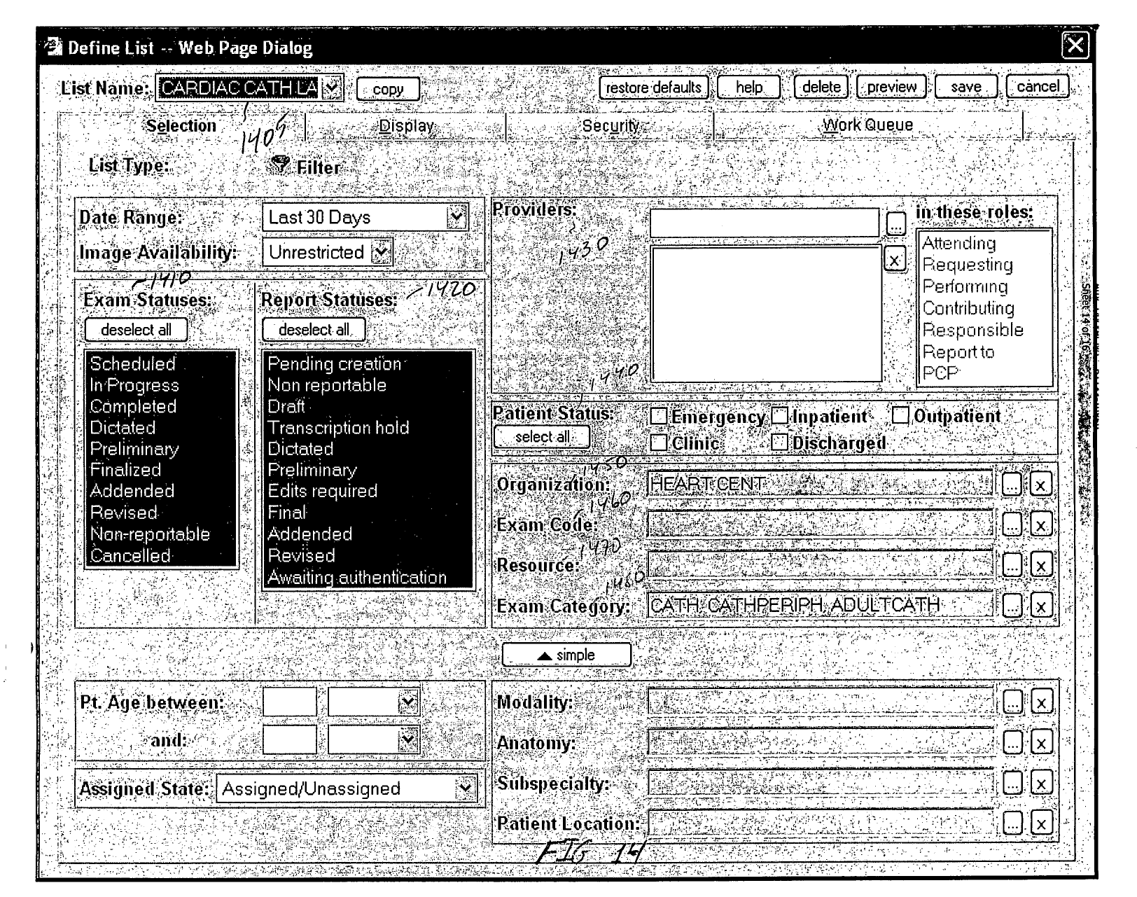

System 100 provides a graphical user interface somewhat similar to that of a conventional PACS, but instead of being limited to user selections pertaining only to images, the user is able to select various aspects of a particular procedure, for example to “drill-down” on details of a portion of a procedure for a particular patient. Referring now to FIG. 14, it provides a user interface that gives a user of system 100 the ability to configure his or her own worklist and filter information as desired. As shown in FIG. 14, a user may select the following selection criteria: exam statuses 1410, report statuses 1420, providers 1430, patient status 1440, organization 1450, exam code 1460, resource 1470, and exam category 1480. For example, in FIG. 14, a user configures a worklist “CARDIAC CATH LAB” 1405. For this worklist, a user selected all exam statuses 1410, all report statuses 1420, “HEART CENT” as organization 1450, and “CATH”, “CATHPERIPH”, and “ADULTCATH” for exam category 1480.

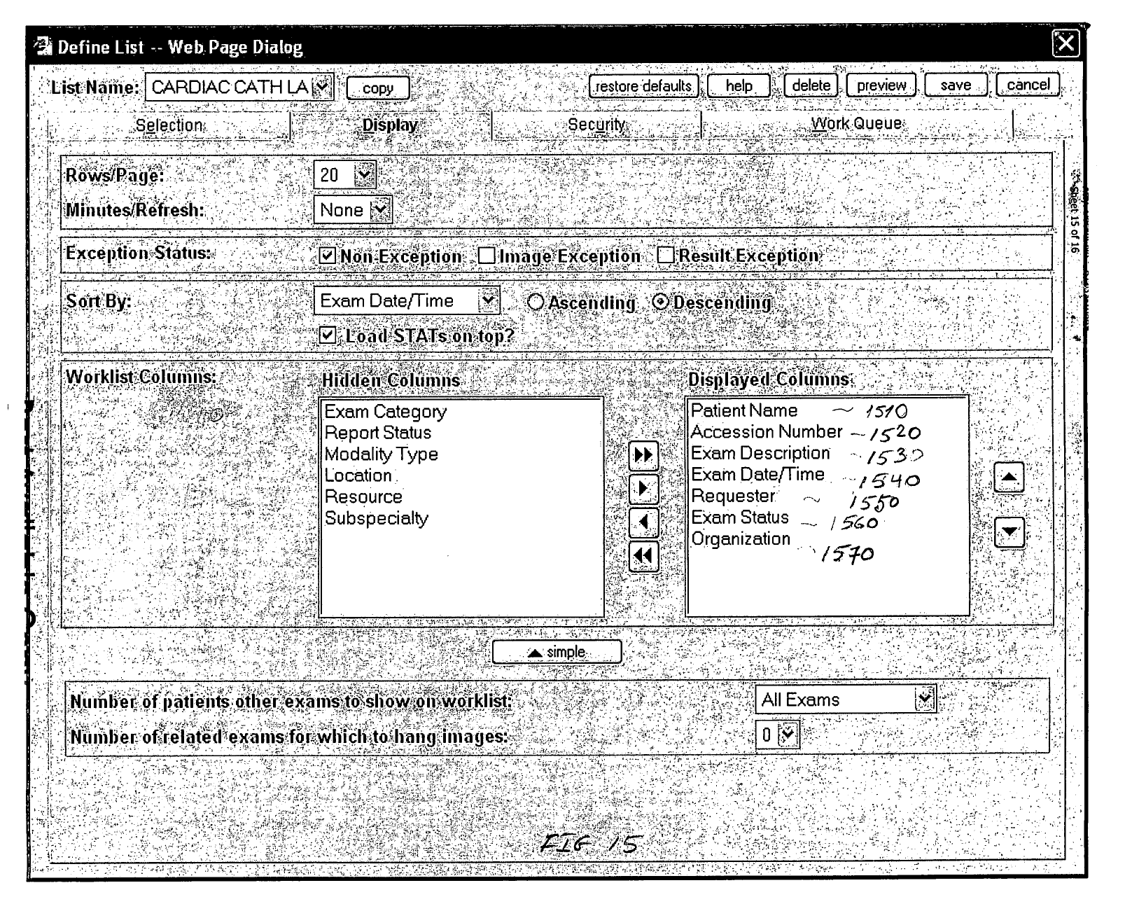

Turning now to FIG. 15, a user interface is shown that allows a user to choose those columns that will be displayed in the worklist. In FIG. 15, a user selected the following columns to be displayed: Patient Name 1510, Accession Number 1520, Exam Description 1530, Exam Date/Time 1540, Requester 1550, Exam Status 1560, and Organization 1570.

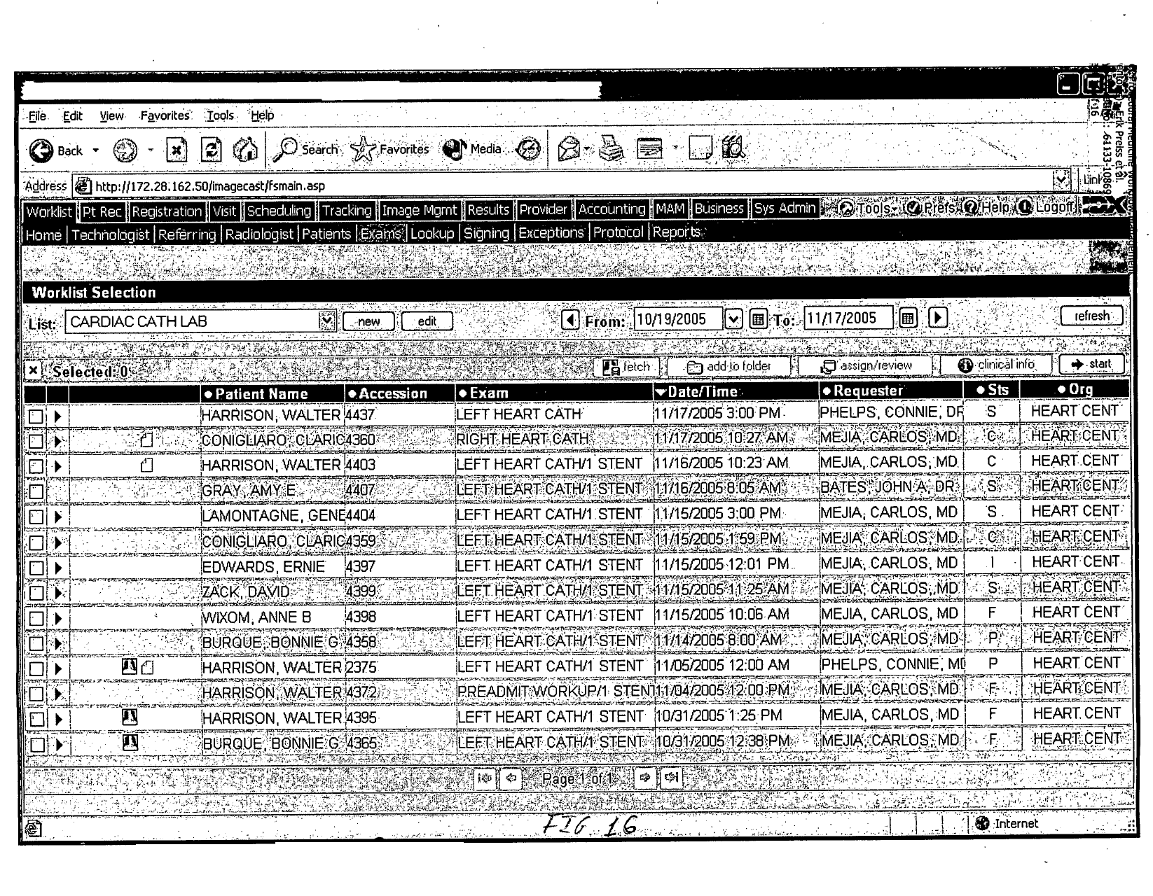

Once the user provided his or her selection criteria, system 100 generates a worklist. Referring now to FIG. 16, it provides a worklist created by a user that allows a user to view, for example, all the reports for Cardiac Cath Lab activity during the period of Oct. 19, 2005 through Nov. 17, 2005. The worklist is created based on the selection criteria chosen by the user.

System 100 provides the user with an interface including checklists, assessments, logging and the like as required for a given procedure. The interface further indicates what work the specialist has that day, again with each selection including correspondences with the details from system 100.

Turning now to more specific examples of the operation of system 100, several categories of its functionality are described below.

Workflow Management

As described above, a fundamental aspect of the operation of system 100 is in the management of workflow associated with procedural medicine. The subtasks to be performed in any given medical procedure vary from procedure to procedure, but the examples listed below address many typical subtasks of common procedures.

Create Diagnostic Report /Document Findings

One of the most common tasks in procedural medicine is documentation of the procedure's details and interpretation of what the procedure's output (e.g., images) indicates from a diagnostic and/or therapeutic perspective. In a typical scenario using system 100, the system first displays any data pertinent to the procedure that has already been collected. The user then enters (either manually or through connection with associated medical devices) and modifies the data associated with performing the procedure, and system 100 records the new data. The user then enters (again, in any convenient manner, from keyboard entry to voice recognition) a summary or interpretation of the data, which the system records. In accordance with good clinical practice, the user then verifies (e.g., signs) the summary/interpretation, and the system 100 distributes, as appropriate, a textual representation of the summary/interpretation.

Capture Dictation

Commonly, physicians and other caregivers find it far more convenient to enter patient information by dictation rather than handwritten notes or keyboard entry. In a preferred embodiment, system 100 captures details of a procedure and the interpretation of images acquired during the procedure via dictation, resulting in little change to the caregiver's current workflow practices. The user first identifies the procedure that was just performed or for which the user is viewing images; system 100 responds with a confirmation of patient information; the user then records interpretations and findings by speaking into conventional digital recording circuitry and software (not shown); and the system stores the recorded information for later retrieval and recognition/transcription. In an alternate embodiment, system 100 converts the speech to text in real time and displays it so that the user can immediately review, correct, and confirm the generated text, at which time system 100 stores the generated text as well as, or instead of, the original voice recording.

Record Textual Findings

In some instances, good clinical practice requires the caregiver to enter information in textual form rather than by dictation. In these situations, system 100 captures the text-input details of a procedure and the interpretation of the images acquired during the procedure. First, the user identifies the procedure for which the interpretation is being provided. System 100 responds with confirmation of patient information. The user then enters the interpretations/findings via typing, and system 100 stores such information for later retrieval. In an alternate embodiment where the user is a transcriptionist performing manual transcription of a caregiver's dictation, the user listens to the corresponding voice file, and enters the appropriate written text based on the voice file.

Enter/Edit Structured Elements

Commonly, it is desirable for caregivers to use coded data sets to record the details of a procedure and the interpretation of images. Use of coded data facilitates business productivity enhancement and leads to increased levels of patient care due to consistency in reporting over time and across a range of caregivers. System 100 manages workflow in this aspect through automatic generation of a textual report from codified elements. Specifically, a user identifies a procedure that was just performed or for which the user is viewing images; system 100 responds with confirmation of patient images; the user records the interpretation/findings using “point-and-click” input on codified data elements; the system 100 stores the codified elements for later retrieval and generates a textual report based on the selected elements.

Distribute Diagnostic Report/Findings

Another near-universal task in procedural medicine workflows is distribution of the results (i.e., findings, interpretations) of the procedure to the requesting or referring health care provider. Typically, that provider will use those results for further diagnosis. System 100 manages distribution of diagnostic reports by identifying the referring provider once the results are known, determining a preferred method of report receipt for that provider (e.g., e-mail, printed report), and transmitting the report by the preferred method. Once the provider reads the diagnostic report, the provider may determine the next course of care for the patient (e.g., schedule follow-up appointment) and enter that event into the workflow via system 100.

Manage Work To Do (Transcription)

Inefficiency prevalent in many health facilities involves identification of work that needs to be done so that when resources become available they can handle that work. As an example, a limited pool of transcriptionists is typically available for an entire facility, and the efficiency of the pool is maximized if there is a convenient way for transcriptionists to know whenever there is work for them to do. System 100 facilitates such efficiency by automatically maintaining a list of voice files that have not yet been transcribed. Specifically, system 100 displays a list of dictations that do not have textual reports. The user (i.e., transcriptionist) selects one of those to work on. System 100 then marks that item as being in use so that it cannot be selected by another transcriptionist. The system then plays the associated voice file and it is transcribed as described above in the “Record Textual Report” workflow description.

Reporting Framework

Aside from workflow management, system 100 provides reporting frameworks to facilitate the various reports commonly associated with medical procedures. Examples for a preferred embodiment are described here.

Identify Patient/Procedure

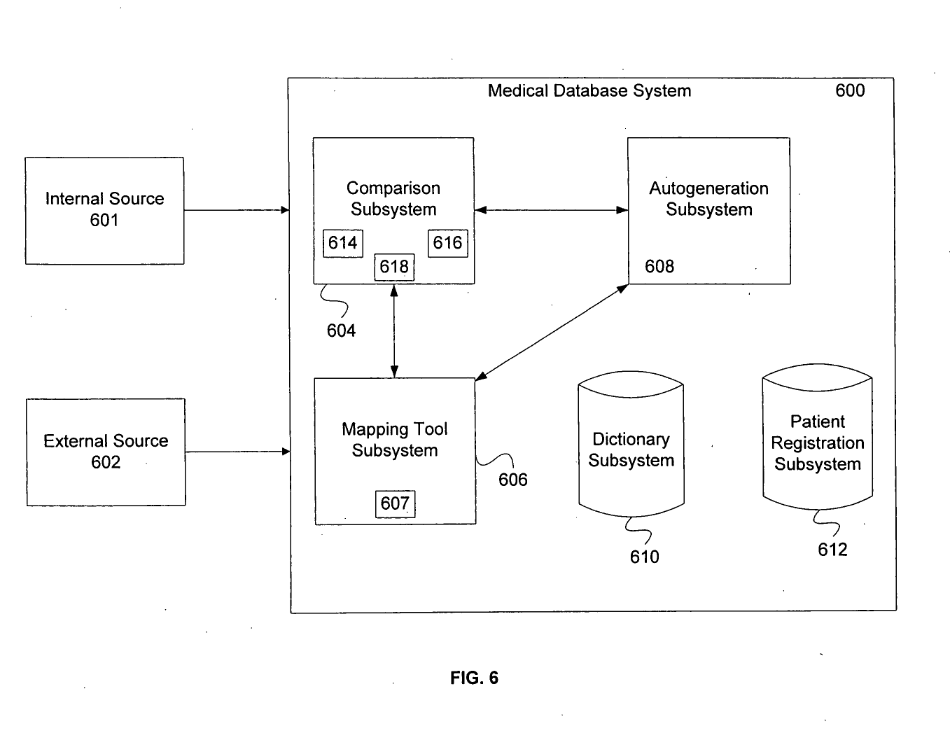

Workflow for virtually any imaginable medical procedure requires identifying both the patient involved and the specific procedure to be performed. In a preferred embodiment, system 100 allows the user to efficiently call up such information for purposes of further data entry or information review. In prior non-computerized systems, this activity typically calls for location of patient files and association of those files with the appropriate requisition of services. In a preferred embodiment, the user, for instance a physician, enters any available identifying information and system 100 responds with a list of possible matches. Such identifying information includes patient identification information, procedure information (e.g., a list of all pending MRI requests for the day), and other information that could help to sort within available databases to identify a particular patient or procedure. Further details on such automatic generation of patient information are provided below in connection with FIGS. 6-13. The physician then selects from the list presented by system 100 the appropriate item, and system 100 responds by displaying pertinent information for the selected patient or procedure.

Create Report/Capture Findings

In existing systems, it is common to keep only the results of a procedure that are pertinent to the diagnosis needed at the time. For instance, imaging for purposes of cardiology may be intended to generate a result concerning blood flow around the heart, and that may be all the information reported as a result of the procedure. However, such an imaging procedure may also incidentally capture other details that could be used in the future for diagnosis of later ailments. In addition, concerns about medical malpractice claims may make it desirable to capture more information about the procedure itself, rather than only the results of the procedure. Accordingly, system 100 facilitates capturing and communicating both procedure results and procedure details. In a preferred embodiment, various mechanisms are made available for capturing this information, based on efficiencies of a particular procedure and caregiver preferences. Thus, using a tool that provides the user with the greatest productivity, the user captures findings observed during the procedure, or the interpretation of images acquired during the procedure, as may be the case, as well as a summary of recommendations for care. In one preferred embodiment, system 100 presents the user with the current information about the procedure, for example images captured during a procedure. The user views those images, and the system provides a number of structures options of findings that can be captured, number of vessels imaged, number of occlusions found, size and degree of severity of each occlusions, etc. The user then captures an interpretation of findings identified in the images, and captures a suggestion for a plan of action, which may include additional imaging procedures to further define the patient's problem or non-procedure based treatments such as medications. In response, system 100 communicates the captured data to data management subsystem 101 for further processing in accordance with desired workflow, which may include communications of the suggested plan of actions to the referring physician or initiating a pharmacy order in an external system 111. In an alternate embodiment for use while a procedure is underway, a user captures various intermediate steps of the procedure for storage and further communication. For example, a radiologist may capture various stages of imaging during mammography or ultrasound imaging to show that various portions of the body were in fact imaged, even though no ailments were found in those portions. In another alternate embodiment, the capture also includes the user signing off on verbal orders given during the procedure, such as sedations given during a procedure.

Map To Common Vocabulary

It is typical for health care facilities to include various devices and systems for collecting healthcare-related information. Unfortunately, no universal standard is currently in use that keeps such information in a common format. Some devices may conform to one popular standard, while others conform to another standard, and still others may hold data in a vendor-proprietary format. Thus, a common workflow activity is to take data collected from disparate, incompatible sources, and translate such into a common format. In a preferred embodiment, system 100 receives data from such various reporting tools that communicate findings data, and translates such data into a set of common data elements that are directly usable throughout system 100.

Report On Clinical Outcomes

Healthcare facilities can improve both the level of patient care and their overall efficiency by analyzing clinical data gathered on the procedures that they undertake. This information is usable for various purposes, such as registry submissions, patient care analysis, trending analysis, credentialing organization, and other research. In a preferred embodiment, system 100 displays for a user (e.g., a quality analyst or business analyst) possible data elements from which to choose. The user then formulates the desired output, not only for content but also for presentation style and output format. System 100 then compiles the data and statistics as requested for the desired content, and then prints or otherwise presents the data as requested.

Assign Billing/Procedure Codes

Numerous systems exist for automating the process of billing in healthcare facilities, and for using procedure codes to correspond to activities at such facilities. It is common for healthcare facilities to have several such systems in operation, and some procedures may require input to or output from more than one such system. Accordingly, in a preferred embodiment, system 100 coordinates billing and procedure code capture among disparate systems and facilitates assignment of billing codes and procedure codes to a common vocabulary. As a result, regardless of what subsystem generates structured text, once any proprietary data elements are mapped to a common vocabulary the billing and procedure codes are integrated with the procedure in a transparent manner. In a preferred embodiment, mapping is made to individual common vocabulary data elements or specific combinations of common vocabulary data elements, as desired. As a specific example, system 100 determines which common data elements in a set of findings are assigned billing/procedure codes. In one embodiment, system 100 compares the collected data elements to data element/billing code combinations that are configured by a common vocabulary module. System 100 then determines which combinations of common data elements that exist in the findings are assigned billing/procedure codes. System 100 then includes the assigned billing/procedure codes in the documentation for the procedure.

Manage Common Vocabulary

Where a common vocabulary is desired for data elements, and in particular where mappings are made from proprietary clinical data elements to the common vocabulary data elements, from time to time healthcare facilities will need to update the mapping. System 100 facilitates such updating in a manner that allows changes in the mapping as needed within a workflow by defining relationships between proprietary elements in structured reporting tools and elements in a common vocabulary, and defining relationships between common vocabulary data elements, or sets of elements in some cases, and billing and procedure codes. In a preferred embodiment, system 100 displays the proprietary data elements and the common vocabulary elements and allows a user to associate corresponding elements together using conventional graphical user interface mechanisms. System 100 then allows the user to select procedure or billing codes for each common element, or set of elements, and associates them together as well. System 100 then stores the selected associations/relationships to use in the mappings described above. In a preferred embodiment, efficiencies are obtained by system 100 allowing establishment of a default set of mappings ahead of that an administrator can simply modify as needed, rather than having to start from scratch to configure system 100. In an alternate embodiment, provisional relationships are dynamically created when a proprietary data element is sent to the mapper and is not currently associated with a common data element. In that case, system 100 displays the non-mapped value to the user and prompts the user to either verify or select the common data element to which it relates. At that time the relationship is stored for future reference. In this manner, relationships can be built on the fly and need not be managed ahead of time.

Turning now in greater detail to mapping of structured data. Currently, in healthcare information systems there are a number of clinical structured reporting vendors and tools on the market. Each vendor has developed a set of structured data elements that can be used to document a diagnostic or therapeutic result. Some medical governing bodies are working towards standardizing the data elements for their specific specialty. However, that process can often be very time consuming and contentious within one specialty, not to mention the effort that would be required to standardize and gain consensus across specialties. Therefore, the problem of being able to compare data and medical outcomes across disparate clinical structured reporting systems will be an issue for the foreseeable future.

System 100 allows hospital staff to use a range of clinical reporting tools and still have the ability to compare the clinical outcomes regardless of the reporting tool. This allows for the end user to select the clinical reporting tool that best suits the clinical domain and/or end user workflow, therefore increasing user productivity and patient care.

System 100 is configurable to map data elements from different reporting tools within the same medical specialty or from tools across specialties or both.

Some of the existing clinical structured reporting tools currently on the market are providing a mapping of their proprietary data elements to a generic set of codes (SNOMED for example). These systems are only looking to solve the issue at the most granular level and are not taking into consideration the fact that some healthcare institutions will have multiple clinical structured reporting systems. In this scenario there will still be clinical data gathered within the institution that cannot be compared to each other, potentially even within the same specialty.

In a preferred embodiment, much of the functionality described above is implemented in a conventional manner, as will be evident to one skilled in the art. One area not conventionally implemented, however, is the automatic generation of patient-related data mentioned above. A preferred implementation of this area is thus detailed herein. Specifically, referring now to FIG. 6 there is shown is a high-level block diagram illustrating a preferred embodiment of a medical database system 600. The system 600 takes as input various patient-related data from various sources. Some sources, for simplicity and clarity referred to herein as the internal source 601, provide patient-related data in a known, standardized format. As one example, the IDXrad product produced by IDX Systems Corporation of Burlington, Vt., provides patient identifying information conforming to the HL7 ADT protocol and including patient data fields that natively match those used in the medical database system 600. For patient-related information from the internal source 601, the medical database system 600 can immediately and unequivocally identify a patient associated with the data provided by the internal source 601 and update the patient's records and other associated information stored by the medical database system.

The medical database system 600 is also configured to accept as input patient-related data coming from other sources that may not provide such data in a form that perfectly matches that used in the medical database system 600. For simplicity and clarity, such sources are referred to herein as an external source 602. Commonly, data from the external source 602 will include certain patient-related data, but the data will not be in a form that perfectly matches the form used by the medical database system 600, nor may the data be sufficiently complete to allow immediate and certain identification of a patient.

In order to provide the most efficient workflow for healthcare personnel accessing the medical database system 600, the system is adapted to automatically generate patient-related data in response to data received from the external source 602 that cannot be matched with a patient already in the system. The system 600 preferably interprets the data provided by the external source 602 to the best of its capabilities, and automatically generates and populates, to the extent possible, associated records and fields associated with the data received from the external source.

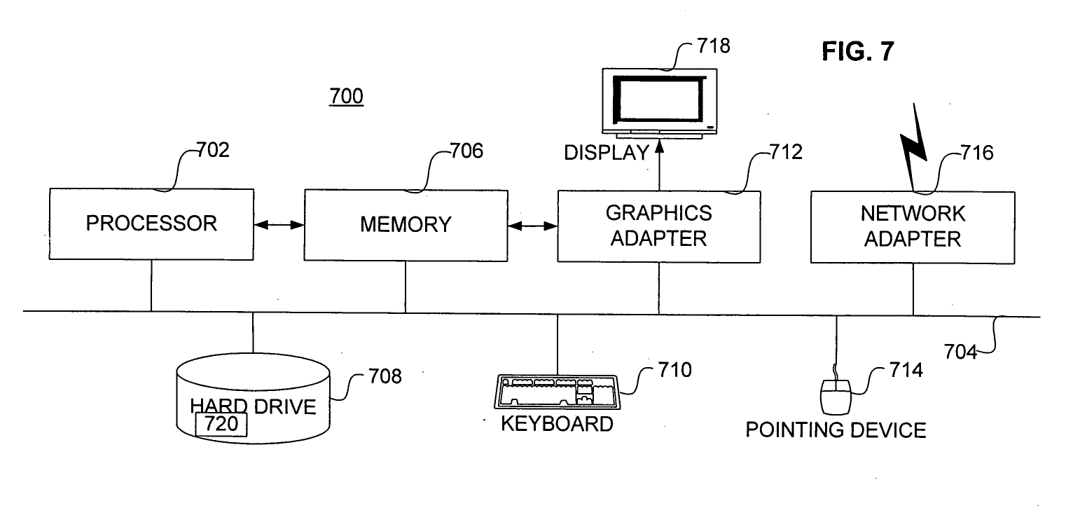

In one embodiment, the medical database system 600 executes on a conventional computer system. FIG. 7 is a high-level block diagram of a computer system for use as the medical database system 600 according to one embodiment. Illustrated are at least one processor 702 coupled to a bus 704. Also coupled to the bus 704 are a memory 706, a storage device 708, a keyboard 710, a graphics adapter 712, a pointing device 714, and a network adapter 716. A display 718 is coupled to the graphics adapter 712.

The at least one processor 702 may be any specific or general-purpose processor such as an INTEL x86 or POWERPC-compatible central processing unit (CPU). The storage device 708 may be any device capable of holding large amounts of data, like a hard drive, compact disk read-only memory (CD-ROM), DVD, or some other form of fixed or removable storage device. The memory 706 holds instructions and data used by the processor 702. The pointing device 714 may be a mouse, track ball, light pen, touch-sensitive display, or other type of pointing device and is used in combination with the keyboard 710 to input data into the computer system 700.

The network adapter 716 couples the computer system 700 to the internal source 601 and external source 602. In one embodiment, the network adapter 716 connects the computer system 700 to a local and/or wide area network, which in turn connects to the internal 601 and/or external sources 602. The network adapter 716 and network may use any conventional networking technology, such as Ethernet, TCP/IP, HTTP, etc. to communicate with the sources 601, 602. In another embodiment, the internal source 601 and/or external source 602 are connected to the computer system 700 through different communication technologies, such as IEEE 1394 FireWire, universal serial bus (USB), serial, and/or parallel connections. In yet another embodiment, there is no direct communication between the sources 601, 602 and the computer system 700 acting as the medical database system 600. Instead, data from the sources 601, 602 are encoded on a storage medium, such as a floppy disk, CD-ROM, DVD, or other magnetic, optical, or semiconductor memory, which is then physically transported, and inserted into, the computer system 700.

Program modules 720 for providing the functionality attributed to the medical database system 600 are preferably stored on the storage device 708, loaded into the memory 606, and executed by the processor 702. Alternatively, hardware or software modules may be stored elsewhere within the computer system 700. As used herein, the term “module” refers to computer program logic utilized to provide the functionality attributed to the modules.

The types of hardware and software within the computer system 700 may vary depending upon the implementation of the medical database system 600. For example, a medical database system 600 operating in a high-volume environment may have multiple processors and hard drive subsystems in order to provide a high processing throughput, as well as multiple displays and keyboards in order to support multiple simultaneous users. Likewise, certain embodiments may omit certain components, such as the display 718, keyboard 710, and/or network adapter 716 depending upon the specific capabilities of the system. In addition, the computer system 700 may support additional conventional functionality not described in detail herein, such as displaying images in a variety of formats, allowing users to securely log into the system 600, supporting administration capabilities, etc.

Returning to FIG. 6, in one embodiment, the medical database system 600 includes various subsystems to perform the functionality of extracting patient-related data received from the external source 602. These subsystems include a comparison subsystem 604, a mapping tool subsystem 606, an autogeneration subsystem 608, a dictionary subsystem 610, and a patient registration subsystem 612. In one embodiment, these subsystems are implemented as modules on the computer system 700.

The mapping tool subsystem 606 receives the data from the external source, interprets, maps, and translates the data, and then calls the comparison 604 and autogeneration 608 subsystems to store the data in the medical database system 600. The comparison subsystem 604 determines if a record corresponding to the data from the external source already exists and, if so, associates the data with the record. If a corresponding record does not already exist, the autogeneration subsystem 608 creates the record and populates the data fields with the received data and other known information.

In one embodiment the data supplied from the internal 601 and external 602 sources include digitized radiology images supplied by a Hospital Information System (HIS), Radiology Information System (RIS), Picture Archive and Communication System (PACS), or another such system. Other embodiments of the system 600 may accept other medical-related data (or even non-medical-related data) instead of or in addition to the radiology images.

In one embodiment, the images can include and/or be accompanied by additional digital data describing the context of the image. In one embodiment, the additional data include patient identifying information, provider identifying information, and/or study identifying information. These data may be encoded in one or more of a variety of formats and protocols, including the Digital Communications in Medicine (DICOM) protocol, the Health Level Seven (HL7) protocol, etc. The data may also include one or more messages in the HL7 or another protocol, including an Admit/Discharge/Transfer (ADT) message, an Orders Message (ORM), a Results Message (ORU), etc. For purposes of clarity, all of these forms of data are occasionally referred to herein as “patient-related data” or simply “patient data.” It should be understood that the functionality of the medical database system 600 can be applied to any types of data. Thus, “patient-related data” and “patient data” are meant to include any types of data with which the described functionality is applicable.

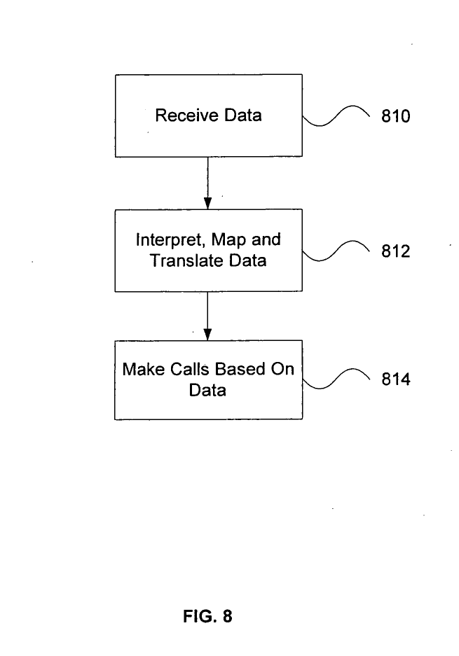

FIG. 8 is a flowchart illustrating the operation of the mapping tool subsystem 606 according to one embodiment of the medical database system 600. Those of skill in the art will recognize that alternative embodiments of the system 600 may perform the illustrated steps in different orders, perform additional steps, or omit certain steps. In a preferred embodiment, the ConnectR product provided by IDX Systems Corporation is used to implement the mapping tool subsystem 606.

The mapping tool subsystem 606 receives 810 the data from the external source 602. In one embodiment, the data relate to a patient, provider, and/or study. The mapping tool subsystem 606 in one embodiment makes a threshold decision of whether the patient-related data contains enough information for the mapping to proceed. If not, one embodiment of the mapping tool subsystem 606 rejects the data and generates an exception (this step is not shown in the figures). The mapping tool subsystem 606 interprets 812 the data related to the patient, provider, and/or exam from the input data and maps it into a representation utilized by the medical database system 600. In one embodiment, the mapping tool subsystem 606 includes an interpretation engine 607 containing rules and logic for interpreting, mapping, and translating data from a variety of external sources in a variety of representations and formats. The interpretation engine 107 uses the rules and logic to interpret the received data and then map the data to the appropriate records, fields, or categories used by the system 600.

The interpretation engine 607 also preferably translates 812 the data, if necessary, from the received format into the format utilized by the system 600. For example, if the data from the external source 602 represents gender as an integer value (e.g., 0=male, 1=female, 2=unknown) and the patient registration subsystem 612 represents gender as an alphanumeric character (e.g., m=male, f=female, u=unknown), the mapping tool subsystem 606 translates the data from the first format to the second format.

The mapping tool subsystem 606 preferably makes application programming interface (API) calls 814 to the comparison 604 subsystem based on the interpreted, mapped, and translated data. In general, the API calls set properties of the dictionary 610 and/or patient registration 612 subsystems to reflect the received data. For example, if the received data describe a patient, the mapping tool subsystem 106 makes API calls to the comparison subsystem 604 causing it to create or update records and/or fields in the dictionary 610 and/or patient registration 612 subsystems in response to the received data. In one embodiment, the logic attributed to the comparison 604 and autogeneration 608 subsystems in the below description is performed by the API calls generated by the mapping tool subsystem 606. In other words, the mapping tool subsystem 606 generates API calls causing the comparison 604 and autogeneration 608 subsystems to perform the method steps and other logic described below.

As illustrated in FIG. 6, the comparison subsystem preferably includes three modules 614, 616, 618. A patient matching module (PMM) 614 preferably utilizes patient identifying information in order to attempt to match the data received in the API call from the mapping tool subsystem 606 with a patient in the patient registration subsystem 612. A provider matching module (PRMM) 616 preferably utilizes provider identifying information in order to attempt to match the data received in the API call with a provider identified in the dictionary subsystem 610. A study matching module (SMM) 618 preferably utilizes study identifying information to match the data received in the API call with an exam stored in the patient registration subsystem 612. If the modules 614, 616, 618 are able to identify a patient, provider, or exam, then the modules update it with the data received in the API call. If the modules 614, 616, 618 are not able to identify the patient, provider, or exam, then the modules preferably call the autogeneration subsystem 608 to create and populate the appropriate records in fields in the dictionary and/or patient registration subsystem 612 for the new patient, provider, or exam.

Turning now to the PMM 614, the PMM 614 examines the received data to determine whether the data relate to an existing patient identified by the patient registration subsystem 612. If the data relate to an existing patient, then the comparison subsystem 604 preferably updates the patient's record in the patient registration subsystem 612 with the newly-received data. Otherwise, the comparison subsystem 604 preferably causes a new patient record to be created in the patient registration subsystem 612 and causes the patient record to be populated with the newly-received data and such additional information as can be determined.

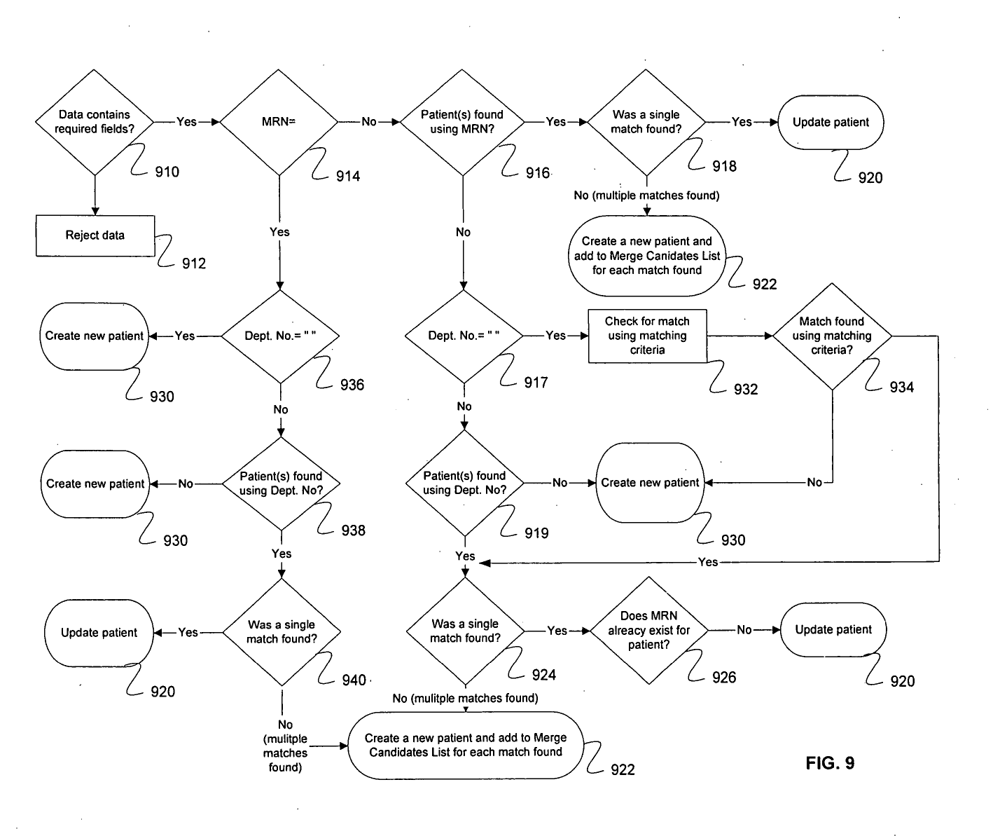

FIG. 9 is a flowchart illustrating the operation of the PMM 614 of the comparison subsystem 604 according to one embodiment of the medical database system 600 in response to receiving data from the external source 602. Those of skill in the art will recognize that alternative embodiments of the system 600 may perform the illustrated steps in different orders, perform additional steps, or omit certain steps.

The PMM 614 preferably initially determines 910 whether the received data contains enough information to support the matching process. In one embodiment, the received data must include at least one of a medical record number (MRN), department number, and master patient index (MPI) number, and a last name/first name pair. Otherwise, the PMM 614 rejects 912 the data and does not attempt to match the data with a patient.

If the data contain enough information to support the comparison process, the PMM 614 determines 914 whether the MRN is specified (i.e., the MRN field is not blank). If the MRN is specified, the PMM 614 determines 916 whether a patient having the MRN is found in the patient registration subsystem 612. If 918 a single matching patient is found in the registration subsystem 612, the PMM 614 causes the patient record in the registration subsystem 612 to be updated 920 with the data. If 918 multiple matching patients are found in the registration subsystem 612, then the PMM 614 preferably creates 922 a new patient record. The PMM 614 also preferably creates 922 a “merge candidates” list containing the new patient record and the multiple matching patients.

If 916 the PMM 614 does not find any patients in the patient registration subsystem 612 matching the MRN, it preferably determines 917 whether a department number is specified in the data. If 917 a department number is specified (i.e., the department number field is not blank), the PMM 614 determines 919 whether a patient having the department number is found in the patient registration subsystem 612. If 924 a single matching patient is found in the registration subsystem 612, the PMM 614 determines 926 whether a MRN already exists for the patient. If 926 no MRN exists for the patient, the PMM 614 preferably causes the patient record in the registration subsystem to be updated 920 with the data received from the external source. If 924 multiple matching patients are found in the registration subsystem 612, or if 926 a MRN already exists for the single matching patient, then the PMM 614 preferably creates 922 a new patient record. The PMM 614 also preferably creates 922 a “merge candidates” list containing the new patient record and the other matching patient records as described previously.

If 919 the PMM 614 does not find a patient using the department number, then the PMM 614 preferably causes a new patient record to be created 930. Thus, if the MRN and department are both specified, but the PMM 614 cannot identify a matching record in the patient registration subsystem 612, the PMM causes a new record for the patient to be created.

If, however, the MRN is specified but does not match a patient, and the department number field is blank 917, the PMM 614 preferably checks for a match using matching criteria 932. In one embodiment, the PMM 614 checks 932 for a match by comparing the last name, first name, date of birth (DOB), social security number (SSN), MRN, and other identification information with information in the patient registration subsystem. Each of these components is weighted as follows:
Last Name=0.5
First Name=0.25
DOB=1.0
SSN=1.0
MRN=3.0
Other ID=3.0
   The PMM 614 declares 934 a match if the total of the weights of any matching data is 3.75 or higher. If 934 one or more matching patients are found, the PMM 614 flow preferably moves to step 924. If the PMM 614 does not find a matching patient, the PMM preferably causes 930 a new patient record to be created.

Returning to step 914, if the MRN field is blank, the PMM 614 preferably determines 936 whether the department number field is also blank. If 936 the department number is blank, the PMM 614 preferably causes 930 a new patient record to be created. If 936 the MRN field is blank, but the data contain a department number, the PMM 614 determines 338 whether a patient having the department number is found in the patient registration subsystem 612. If 938 no matching patient is found, the PMM 614 causes 930 a new patient record to be created. If 940 only a single patient is found having a matching department number, the PMM 614 preferably causes the patient record in the registration subsystem to be updated 920 with the data received from the external source. If 940 multiple matching patients are found in the registration subsystem 612 then the PMM 614 preferably returns to step 922 and creates a new patient record. The PMM 614 also preferably creates 922 a “merge candidates” list containing the new patient record and the other matching patient records.

At the end of the process described in FIG. 9, the PMM 614 has either updated 920 a patient record, created 930 a new patient record, or created 922 a new patient record and added it to a list of potential merge candidates. In the latter case, the merge candidate list is preferably revisited to determine whether the new patient record can be merged into one of the merge candidates. In one embodiment, this determination is made by a human user reviewing the patient records.

As described above, the PRMM 616 preferably utilizes provider identifying information in order to attempt to match the received data with a provider identified in the dictionary subsystem 610. As used herein, the term “provider” refers to a doctor or other healthcare provider. However, those of ordinary skill in the art will recognize that the functionality provided by the PRMM 616 can also be used to match data with entities other than providers. Accordingly, the term “provider” should be interpreted to include any other entity for which the functionality of the PRMM 616 is applicable.

The provider data received by the mapping tool subsystem 606 from the external source 602 may be supplied in an HL7 ADT, ORM, or ORU message. If the provider data is supplied in an HL7 message, the data provided to the PRMM 616 via the API call are preferably supplied as a composite name (CN) data type. The HL7 CN data type provides the following components related to the provider: Provider ID (also referred to as the “provider number”), Provider Last Name, Provider First Name, Provider Middle Name, Provider Suffix Name, Provider Prefix Name, and Provider Title Name. The provider ID may identify both the provider and an organization with which the provider is associated. The provider data may also be provided to the PRMM 616 as an HL7 MFU or similar master file/dictionary management message. In addition, the provider data may also be supplied as DICOM header information, preferably using a Person Name (PN) value representation. The DICOM PN value representation supplies the following components: Provider Last Name, Provider First Name, Provider Middle Name, Provider Suffix Name, Provider Prefix Name, and Provider Title Name.

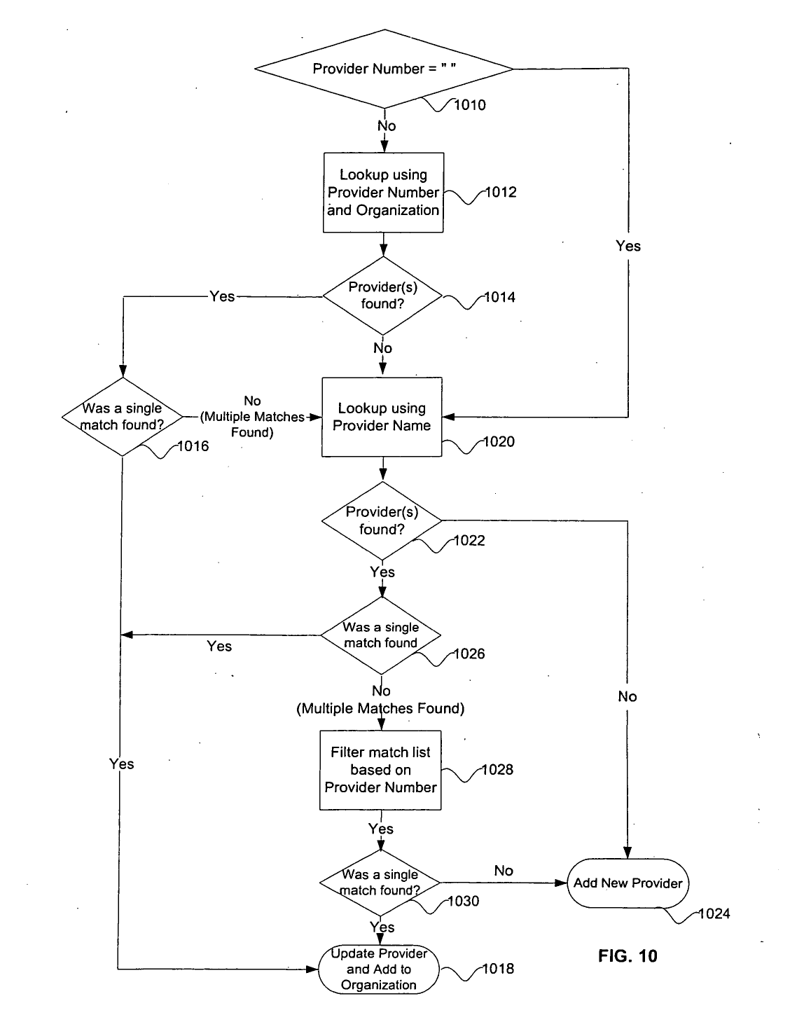

FIG. 10 is a flowchart illustrating steps performed by the PRMM 616 module when performing provider matching processing according to an embodiment of the comparison subsystem 604. In general, if the data received in the API call from the mapping tool subsystem 606 relate to an existing provider, then the PRMM 616 preferably updates the provider's record in the dictionary subsystem 610 with the newly-received data. Otherwise, the PRMM 616 preferably causes a new provider to be created in the dictionary subsystem 610 and causes the provider record to be populated with the newly-received data and such additional information as can be determined. Those of skill in the art will recognize that alternative embodiments of the subsystem 604 may perform the illustrated steps in different orders, perform additional steps, or omit certain steps.

Initially, the PRMM 616 determines 1010 whether the data include a provider number. If 1010 the data specify the provider number, the PRMM 616 looks up 1012 the provider number in the dictionary subsystem 610 to determine 1014 whether it contains a matching provider. If 1016 the dictionary subsystem contains a single provider matching the provider number, the PRMM 616 updates 1018 the provider and/or organization record in the dictionary subsystem 610 as specified by the new data.

If 1010 the provider number is not specified, the provider number is specified but the PRMM 616 did not find 1014 the provider in the dictionary subsystem 610, or if the PRMM 616 found 1016 multiple matching providers in the dictionary subsystem, the PRMM 616 preferably uses the provider name to look up 1020 the provider in the dictionary subsystem 610. If 1022 the PRMM 616 does not find a provider having a matching name in the dictionary subsystem 610, the PRMM adds 1024 the provider to the dictionary subsystem. If 1026 the PRMM 616 finds a single matching provider, the PRMM preferably updates 1018 the provider and/or organization record in the dictionary subsystem 610 as specified by the new data.

If 1026 the PRMM 616 finds multiple matching providers, the PRMM preferably filters 1028 the match list based on the provider number (if available). If 1030 the filtering does not produce a single provider, then the PRMM 616 preferably adds 1024 the provider to the dictionary subsystem 1024. If 1030 the filtering does produce a single provider, then the PRMM preferably updates 1018 the provider and/or organization record in the dictionary subsystem 610 as specified by the new data.

As described above, the SMM 618 preferably utilizes study identifying information to match the received data with an exam stored in the patient registration subsystem 612. As used herein a “study” is a procedure performed on a patient as part of an exam. Multiple studies may be associated with a single exam. For example, an exam may be associated with multiple images, several lab reports, and a set of comments. Each image or group of images, lab reports, etc. can constitute a “study.”

The study data provided by the mapping tool subsystem 606 to the SMM 618 in one embodiment have been extracted from a DICOM header. These data preferably specify one or more of the following fields: patient identifying number such as the MRN, department number, or MPI number; organization code; accession number; scheduled date/time; and exam code. If the accession number and/or scheduled date/time are not provided, these data are preferably generated by the SMM 618. The data may also specify information about a patient, such as first and last names, date of birth, SSN, etc. In general, if an exam corresponding to the study already exists, the SMM 618 updates the exam with the new study. If no exam corresponding to the study exists, the SMM 618 creates a new exam and associates the study with it.

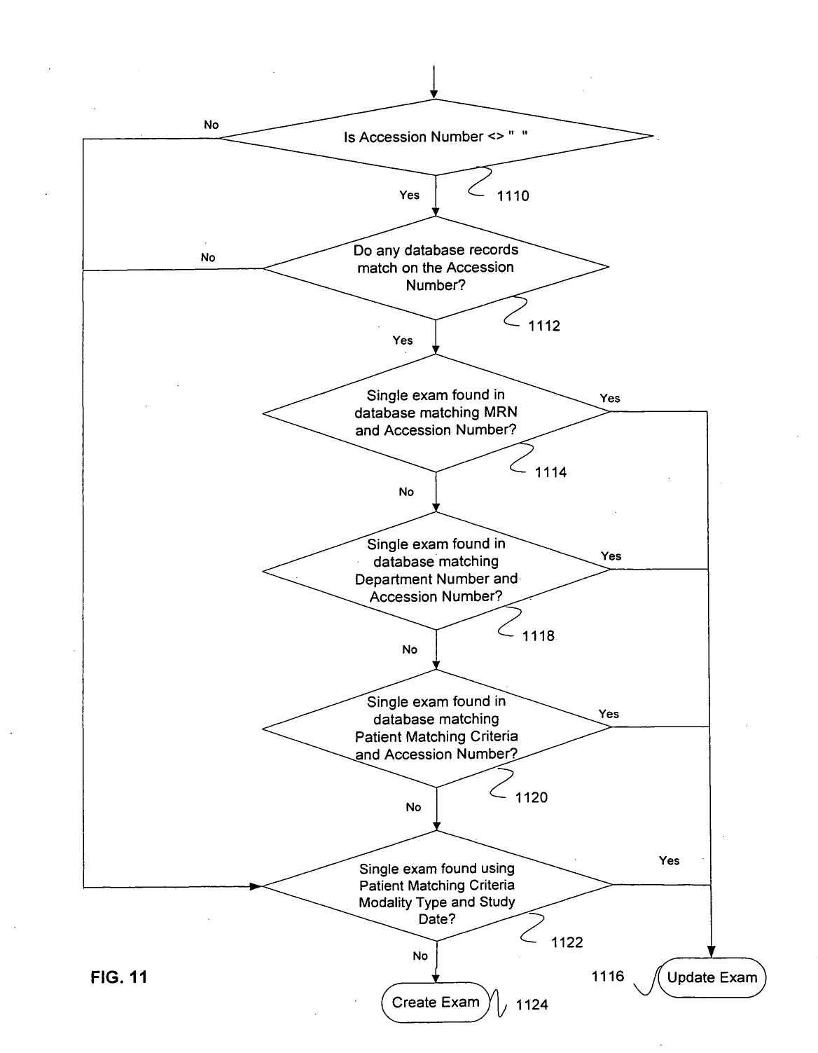

FIG. 11 is a flowchart illustrating steps performed by the SMM 618 module when performing study matching processing according to an embodiment of the comparison subsystem 604. Those of skill in the art will recognize that alternative embodiments of the subsystem 604 may perform the illustrated steps in different orders, perform additional steps, or omit certain steps. Initially, the SMM 618 determines 1110 whether the data specify an accession number. If the data specify an accession number, the SMM 618 determines 1112 whether any exam records in the patient registration subsystem 612 match the accession number.

If 1114 a single exam record in the patient registration subsystem 612 matches the accession number and also matches the MRN, the SMM 618 updates 1116 the exam record with the study data received from the external source 602. If 1118 a single exam record in the patient registration subsystem 612 matches the accession number and also matches the department number, the SMM 618 updates 1116 the exam record with the study data.

If steps 1114 and 1118 have failed to produce a match, the SMM 618 preferably determines 1120 whether a single exam record in the patient registration subsystem 612 matches the accession number and also matches according to patient matching criteria. In one embodiment, the patient matching criteria associates weights to patient information as follows:
Last Name=0.5
First Name=0.25
DOB=1.0
SSN=1.0
   In one embodiment, the patient information received from the external source 602 is compared with the patient information in the exam record having the accession number and the weights of the matching fields are summed. A match is declared if the sum is greater than or equal to 1.75. Alternative embodiments of the system 600 may perform this matching differently. If 1120 there is a match, the SMM 618 updates 1116 the exam record with the study data.

If 1110 no accession number is supplied, or an accession number is supplied but no matching records are found during the previously-described steps, the SMM 618 preferably determines 1122 whether a matching exam record can be found in the patient registration subsystem 612 by considering the patient matching criteria, modality type, and study date. If 1122 there is a match, the SMM 618 updates 1116 the exam record with the study data. Otherwise, the SMM 618 creates 1124 an exam and associates the study with it.

As can be seen from the above-described operation of the comparison subsystem 604, the subsystem in essence operates in two modes. The first mode occurs when the data received from the external source 602 follows the HL7 standard. In this case, the comparison subsystem 604 finds a patient, provider, or exam based on the MRN or another reliably unique identifier like the department number. If a patient, provider, or exam is found based on this information, no other matching is required, and the associated database record is updated with any new information contained in the data from the external source 602.

The second mode occurs when the data is not from an HL7 compliant source. In this case, the comparison subsystem 604 finds a patient, provider, or exam by assigning weights to certain fields, such as the names, the DOB, SSN, MRN, etc. Thus, in the second mode the comparison subsystem 604 is able to identify a matching record based upon somewhat ambiguous information.

The mapping tool subsystem 606 preferably calls on the autogeneration subsystem 608 to automatically generate and populate records and fields in the dictionary 610 and patient 612 registration subsystems. In one embodiment, the autogeneration subsystem 608 executes in response to the comparison subsystem 604 reaching a processing state where it updates or creates one or more records or fields. These processing states include the “update patient” 920 and “create new patient” 930 steps in FIG. 9, the “update provider and add to organization” 918 and “add new provider” 924 steps in FIG. 9, and the “update exam” 1016 and “create exam” 1024 steps in FIG. 10.

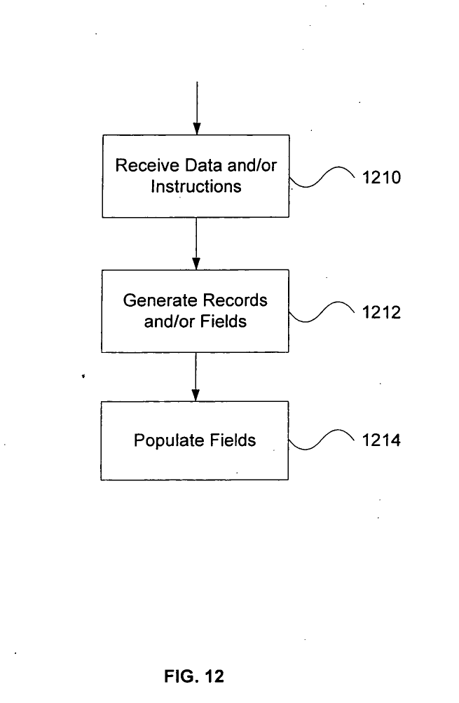

FIG. 12 is a flowchart illustrating the operation of the autogeneration subsystem 608 according to an embodiment of the medical database system 600. Those of skill in the art will recognize that alternative embodiments of the subsystem 608 may perform the illustrated steps in different orders, perform additional steps, or omit certain steps. The autogeneration subsystem 608 receives 1210 an API call from the mapping tool subsystem 1206 containing data and/or instructions indicating a new record or field(s) to be created or an existing record or field(s) to be updated. In another embodiment, the autogeneration subsystem 1208 receives the API call from the comparison subsystem 1204.

The autogeneration subsystem 608 generates 1212 the records and/or fields in the dictionary 610 and/or patient registration 612 subsystems. In general, each record in the dictionary 610 and patient registration 612 subsystems has a set of associated fields for storing data associated with the record. In one embodiment, the set of fields in each record depends upon the type of record. In other embodiments, other factors or information may control the number and types of fields associated with each record. Each field can hold data in the form of numeric, textual, and/or binary information, and/or any other data type adapted for storage in a database. For example, fields may indicate the name, age, and provider associated with a patient. In addition, fields may store radiographic or other images associated with the patient.

The mapping tool subsystem 606 preferably instructs the autogeneration subsystem 608 to populate 1214 the appropriate fields with the appropriate data. The mapping tool subsystem 606 preferably causes the autogeneration subsystem 608 to place the interpreted, mapped, and/or translated data from the external source 602 into the appropriate field or fields. The autogeneration subsystem 608 also preferably populates 1214 certain fields with data derived from other fields in the dictionary 610 and/or patient registration 612 subsystems. For example, the data received from the external source 602 may have relationships with, or trigger certain dependencies based on, data in the dictionary 610 and/or patient registration 612 subsystems that cause certain other fields to take on certain values. In addition, the autogeneration subsystem 608 preferably populates 1214 certain fields with default values. The fields are described in more detail below, and any and all of the fields may be populated with default or other values depending upon the embodiment of the system 600. This step is advantageous because it creates substantially complete records in response to the receipt of limited data.

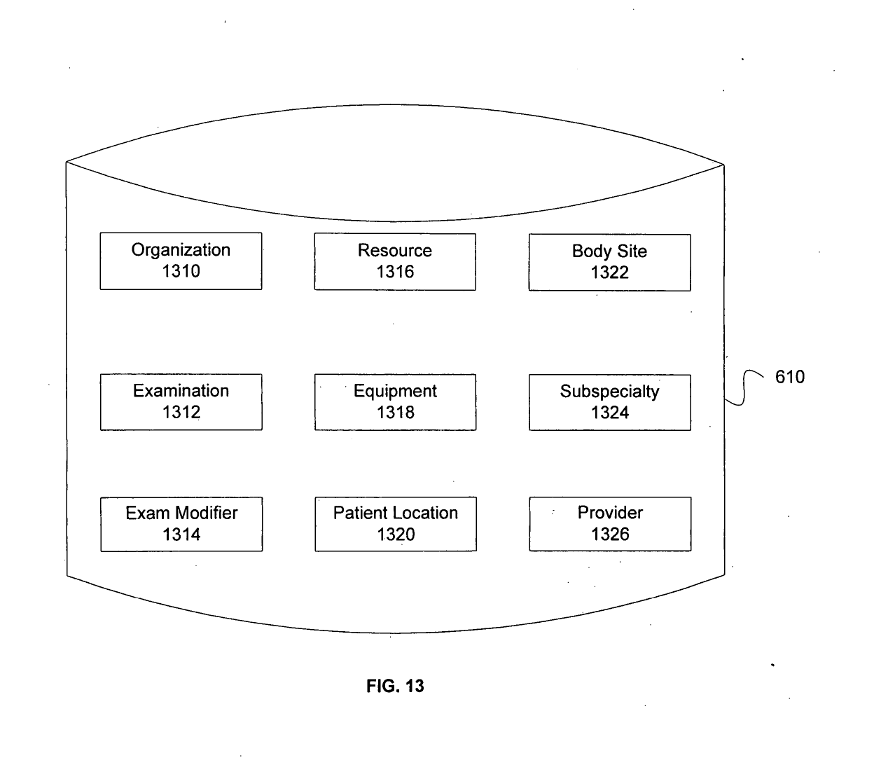

FIG. 13 is a block diagram illustrating a more detailed view of the dictionary subsystem 610. In one embodiment, the dictionary subsystem 610 is implemented through conventional database technology. In a preferred embodiment, the dictionary subsystem contains nine logically separate dictionaries: organization 1310, examination 1312, exam modifier 1314, resource/resource group 1316 (referred to herein as the “resource” dictionary), equipment 1318, patient location 1320, body site 1322, subspecialty 1324, and provider 1326. Alternative embodiments of the subsystem 610 omit one or more of these dictionaries and/or include other dictionaries.

In one embodiment, each record in the organization dictionary 1310 includes fields for an organization code and a description. The organization code indicates the organization that provided the information associated with the entry. The description field describes the organization. In one embodiment, the organization code is a required field and an organization dictionary record is not generated unless this code is known.

Each record in the examination dictionary 1312 preferably includes fields for an organization code, examination code, description, modality type, body site, and subspecialty. In one embodiment, the organization code and examination code are required fields and an examination dictionary record is not generated unless these codes are known.

Each record in the exam modifier 1314 dictionary preferably includes fields for an organization code, an exam modifier code, a description, a modality type, a body site, and a subspecialty. In one embodiment, the organization code and exam modifier code are required fields and an exam modifier dictionary record is not generated unless these codes are known.

Each record in the resource dictionary 1316 preferably includes fields for an organization code, a resource code, a description, and a modality type. In one embodiment, the organization code and resource code are required fields and a resource dictionary record is not generated unless these codes are known.

Each record in the equipment dictionary 1318 preferably includes fields for an organization code, an equipment code, an equipment type code, a description, and a modality type. In one embodiment, the organization code, equipment code, and equipment type codes are required fields and an equipment dictionary record is not generated unless these codes are known.

Each record in the patient location dictionary 1320 preferably includes fields for a patient location code and a description. In one embodiment, the patient location code is a required field and a patient location dictionary record is not generated unless this code is known.

Each record in the body site dictionary 1322 preferably includes fields for a body site code and a description. In one embodiment, the body site code is a required field and a body site dictionary record is not generated unless this code is known.

Each record in the subspecialty dictionary 1324 preferably includes fields for a subspecialty code and a description. In one embodiment, the subspecialty code is a required field and a subspecialty dictionary record is not generated unless this code is known.

Each record in the provider dictionary 1326 preferably includes fields for an organization code, a provider number, a provider last name, a provider first name, a provider middle name, a provider suffix name, a provider title name, a “can interpret” flag, and an “is technologist” flag. In one embodiment, the organization code, provider last name, and provider first name are required fields and a provider dictionary record is not generated unless these data are known.

As with the dictionary subsystem 610, the patient registration subsystem 612 is preferably implemented through conventional database technology. In one embodiment, the patient registration subsystem 612 stores information about patients, exams, and studies. As such, the patient registration subsystem 612 may contain information that complements or overlaps information stored in the dictionary subsystem 610. In one embodiment, the patient registration subsystem 612 and dictionary subsystem 610 are logically separate modules in a single database system.

The above description is included to illustrate the operation of the preferred embodiments and is not meant to limit the scope of the invention. From the above discussion, many variations will be apparent to one skilled in the relevant art that would yet be encompassed by the spirit and scope of the invention.

What is claimed is:

1. A procedural medicine workflow system, comprising:
a data management subsystem configured to capture data corresponding to a medical procedure;
a span of procedure subsystem configured to capture and track information corresponding to selected portions of the medical procedure;
a work organization subsystem coupled to the data management subsystem and the span of procedure subsystem, configured to select appropriate resources for performing the procedure and to present subsets of the captured data to the selected resources as needed for the resources to perform the procedure; and
a business management subsystem coupled to the work organization subsystem and configured to monitor business and patient care aspects of the resources and the procedure.

2. The system as in claim 1, wherein the data management subsystem receives a portion of the data from a digital medical imaging apparatus.

3. The system as in claim 1, wherein the data management subsystem receives a portion of the data from a radiology imaging device.

4. The system as in claim 1, wherein the data management subsystem receives a portion of the data from a cardiology imaging device.

5. The system as in claim 1, wherein the data management subsystem receives the data from multiple imaging components.

6. The system as in claim 1, wherein the data management subsystem integrates the data from a subset of medical devices, electronic medical records, scanned documents, database web services and gathered structural clinical data.

7. The system as in claim 1, wherein the information captured and tracked by the span of procedure subsystem includes at least one of insurance information, medical orders, medical history, scheduling, and tracking information.

8. The system as in claim 1, wherein the work organization subsystem is further adapted to filter the data for presentation to one of the resources so that only a portion of the data pertinent to the one resource in the context of the procedure is presented.

9. A method performed by a procedural medicine workflow system, the method comprising:
capturing data corresponding to a medical procedure;
capturing and tracking information corresponding to select portions of the procedure;
selecting appropriate resources for performing the procedure and presenting subsets of the data to the selected resources as needed for the resources to perform the procedure; and
monitoring business and patient care aspects of the resources and the procedure.

10. The method of claim 9, wherein capturing data further comprises obtaining data from a digital medical imaging apparatus.

11. The method of claim 9, wherein capturing data further comprises obtaining data from a radiology imaging device.

12. The method of claim 9, wherein capturing data further comprises obtaining data from a cardiology imaging device.

13. The method of claim 9, wherein capturing data includes obtaining data from multiple imaging components.

14. The method of claim 9, wherein capturing data further comprises integrating data from a subset of medical devices, electronic medical records, scanned documents, database web services and gathered structural clinical data.

15. The method of claim 9, wherein the information captured and tracked includes at least one of insurance information, medical orders, medical history, scheduling, and tracking information.

16. The method of claim 9, further comprising filtering the data for presentation to one of the resources so that only a portion of the data pertinent to the one resource in the context of the procedure is presented.

17. A computer program product comprising:
a computer-readable medium having computer program code embodied therein for performing procedural workflow management, the computer program code adapted to:
capture data corresponding to a medical procedure;
capture and track information corresponding to select portions of the procedure;
select appropriate resources for performing the procedure and presenting subsets of the data to the selected resources as needed for the resources to perform the procedure; and
monitor business and patient care aspects of the resources and the procedure.

18. A procedural medicine workflow system, comprising:
a registration database configured to store a plurality of medical records containing patient-related data;
a mapping module configured to receive patient-related data and to map the patient-related data into a representation utilized by the medical database system;
a comparison module, coupled to the mapping module, configured to receive the mapped patient-related data and to determine whether the mapped patient-related data matches a medical record stored in the registration database; and
an autogeneration module, communicatively coupled to the mapping module and the comparison module, configured to generate one or more medical records and to populate the one or more medical records with the received patient-related data, responsive to the patient-related data not matching the medical record stored in the registration database.

19. A method performed by a procedural medicine workflow system, the method comprising:
storing a plurality of medical records containing patient-related data;
receiving the patient-related data and mapping the patient-related data into a representation utilized by a medical database system;
receiving the mapped patient-related data and determining whether the mapped patient-related data matches a stored medical record; and
generating one or more medical records and populating the one or more medical records with the received patient-related data, responsive to the patient-related data not matching the stored medical record.
